(19) United States
(12) Patent Application Publication
(10) Pub. No.: US 2021/0100533 A1
(43) Pub. Date: Apr. 8, 2021

(54) SYSTEMS AND METHODS FOR ANALYSIS OF URINE AND FECAL MATTER

(71) Applicant: 11 Health and Technologies Inc., Irvine, CA (US)

(72) Inventors: Michael Seres, Radlett (GB); Anupriya Jai Tilak Naik, Irvine, CA (US); David Ramirez-Ayala, Baldwin Park, CA (US); Bryan Went, Camarillo, CA (US); Erick Went, Camarillo, CA (US); Tianbin Zhao, Irvine, CA (US); Yumeng Wu, Irvine, CA (US); Robert Fearn, Irvine, CA (US); Sabrina Kaefer, Irvine, CA (US); Balaji Meda Ravindra, Los Angeles, CA (US)

(21) Appl. No.: 17/065,123

(22) Filed: Oct. 7, 2020

Related U.S. Application Data
(60) Provisional application No. 62/912,527, filed on Oct. 8, 2019.

(51) Int. Cl. A61B 10/00; A61B 5/00; A61B 5/20; A61B 5/1477; A61B 5/145; A61F 5/44; A61B 5/1473; A61F 5/441; A61F 5/445

(52) U.S. Cl. CPC A61B 10/0038; H04W 88/02; A61B 5/208; A61B 5/6811; A61B 5/6852; A61B 5/1477; A61B 5/14532; A61B 5/14546; A61B 5/14507; A61F 5/4404; A61B 5/746; A61B 5/1473; A61F 5/441; A61F 5/445; A61B 2562/16; A61B 2560/0214; A61B 5/0022

(57) ABSTRACT

A miniaturized monitoring device can include electronic circuitry, housing, and communications protocols. The device can be a robust sensing device that can quantify electrolyte content accurately and transmit continuously from within fecal effluent or urine, or other human bodily fluid and/or solid samples. The device can be fully immersed in the testing sample, which can be in a container, an ostomy bag, or otherwise.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims benefit of U.S. Provisional Patent Application No. 62/912,527, filed on Oct. 8, 2019, the entirety of which is hereby incorporated by reference and made part of this specification.

### BACKGROUND

Metabolism and/or digestion of food can produce waste products such as water, salt, urea, uric acid, solid and/or semisolid waste materials, and/or others. Accumulation of these wastes can be harmful to the human body. The human body can remove the wastes, for example, in the form of urine and feces.

Electrolyte analysis of human fluid samples currently utilize Flame Emission Photometers (FEP) or more Ion-Selective Electrodes (ISE). However, current methods require centralized equipment based within clinical laboratories. A patient is typically instructed to collect all urine for a 24-hour period, under specific directions provided by the lab. Upon completion of the process, the samples should be returned to the lab for processing. Results tend to be available within 1-2 days. 24-hour samples are generally required because the amount of sodium in urine can vary throughout the day, leading to concerns that a single sample may be an inaccurate reflection of average sodium levels.

### SUMMARY

Although 24-hour urine collection is considered the gold standard by agencies such as the WHO (World Health Organization), the Pan-American Health Organization (PAHO) and the US Centre for Disease and Control Prevention (CDC), there is emerging consensus that casual or ‘Spot’ urine sampling can provide valuable data.

ISEs are able to detect the specific ions of interest through the application of a selective membrane at the ISE, which selectively allow the passage of the ions of interest. At equilibrium, the potential difference that exists between two electrodes of an ISE is governed by the concentration of the test solution described by the Nernst equation. This potential difference measured is proportional to the logarithm of the concentration. The Nernst equation demonstrates that the relationship between the potential difference and ion concentration can be determined by measuring the potentials of at least two solutions of known concentration resulting in a plot based on the measured potential and logarithm of the ion concentration. Based on this plot, the ion concentration of an unknown solution can be determined by measuring the potential and corresponding it to the plot.

As described above, current methods for electrolyte analysis of human fluid samples require a 24-hour urine collection and at least 1-2 days of wait for the lab results. More sophisticated approaches are needed for biomarker analysis in biological fluids in order to provide real time data that is more easily accessible to patients and their clinicians and caretakers. The present disclosure provides devices designed for measuring the real-time trends in changing concentrations of the electrolytes, such as sodium and potassium, in human fluid samples over time. The device can be an electrochemical sensing device. The device can additionally provide analytics of inflammatory markers such as protein, blood, leukocytes, nitrites, and/or more complex biomolecules such as fecal calprotection (FCP) and c-reactive protein (CRP).

ISEs typically require a large form factor analyzer, for example, the Altair 240 Automated Chemical Analyzer, the Excel Semi-Automated Chemical Analyzer, Thermo Scientific Gallery Discrete Analyzer, or other benchtop chemical analyzers, with carefully calibrated circuitry to provide reliable readings. In the present disclosure, the sensing device is miniaturized to be used at the point of care within the form factor described herein (for example, with a device casing not exceeding about 30 mm in diameter and/or about 10 mm in height). The device disclosed herein can include an electronic circuit board onto which the ISEs can be mounted directly. The electronic circuit board can further mount a microprocessor, a wireless communication module (for example, a Bluetooth module), an antenna, amplifiers, battery, and other electronic components. The device can also include a temperature sensor, which can allow for temperature compensation calculations in the event of a fluctuating environmental temperature, which may affect the measurements of the device. Firmware can control the device lifecycle, sampling, data conversion, data encryption, and/or transmission.

The present disclosure provides a self-contained analysis system which can be placed within an effluent container. The self-contained analysis system can measure parameters associated with a fluid of a patient, such as Sodium, Potassium, Glucose, Lactate, or the like, some combination thereof as well as analyzing their trends over time.

The system can include an immersible, fully wireless bioanalytical sensing device with built-in electronics. The sensing device and the measured parameters can be readily and remotely accessible to patients, clinicians, caretakers, and/or others for use at the point of need. The sensing device can be a miniaturized device encased in a smaller form factor (for example, with a device housing not exceeding about 30 mm in diameter and/or about 10 mm in height) that can be safely immersed in urine, fecal effluent, or other bodily fluid and/or solid samples. Active components of the device can be protected from the hostile environment of human effluent, for example, by a casing and/or one or more sealing components. The sensing device can be capable of communicating with a smartphone or another receiver device. The communication between the sensing device and the receiver device can be established when the sensing device is within a predetermined distance from the receiver device. For example, the wireless communication technology can be Bluetooth, for example, Low Energy Bluetooth. The sensing device can output readings of the electrolyte content of that sample in real time. In this disclosure, “real time” should be understood to include processing time of the electronics in the sensing device.

The self-contained a monitoring device can comprise one or more sensors configured to detect parameters associated with a fluid of a patient, a wireless transmitter, and a housing. The housing can be configured to enclose the power source, the one or more hardware processors, and the wireless transmitter, and to position the one or more sensors to be in contact with the fluid. The housing can be configured to allow the system to be removably placed within the effluent container. The wireless transmitter can be configured to transmit sensor data to an external device. The housing can include a bottom portion and a top portion. The bottom portion can include a bottom wall and at least one side wall. The top portion can include a first side configured to receive the at least one sensor and a second side configured to removably couple to the bottom portion to form a watertight cavity. The cavity can be configured to receive at least one of the power source or the one or more hardware processors. The one or more sensors can be fully enclosed within the waterproof casing. The one or more sensors can be partially enclosed within the waterproof casing and partially extending away from the casing. The effluent container can include an ostomy bag, urinary catheter, specimen container, or toilet bowl. The fluid of a patient can include at least one of urine or fecal waste. The monitoring device can be enclosed in a waterproof casing. The monitoring device can include a filter configured to filter solids from the fluid of the patient. The monitoring device can include one or more microfluidic channels configured to transmit the fluid to the at least one sensor. The system can include an agitator configured to agitate the fluid. The agitator can be configured to cause the fluid to pass through the at least one sensor. The at least one sensor can include an electrochemical sensor configured to measure at least one of sodium, glucose, or potassium. The one or more hardware processors can be configured to transmit a signal based on the parameters. The signal can be a Bluetooth signal. The power source can include a rechargeable battery. Optionally, the power source can include an AC power source. The monitoring device can be configured to move freely within the effluent container. The monitoring device can be configured to be placed without attachments in the effluent container. The monitoring device can be configured to be placed into the effluent container and to move about the effluent container.

The self-contained system may analyze the contents of an ostomy bag. The self-contained analysis system can include an ostomy bag and a monitoring device. The monitoring device can include at least one sensor configured to detect parameters associated with effluent contained in the ostomy bag and a housing. The housing can be configured to enclose a power source, one or more hardware processors, and a wireless transmitter, and to position the one or more sensors to be in contact with the effluent. The housing can be configured to allow the system to be placed within the ostomy bag. The wireless transmitter can be configured to transmit sensor data to an external device. The at least one hardware processor may be in communication with the at least one sensor and may be configured to: receive sensor data from the at least one sensor; determine at least one effluent parameter based on the sensor data; analyze the at least one effluent parameter to determine a parameter characteristic; and transmit an alert associated with the sensor data to a clinician device based on the parameter characteristic. The at least one sensor can be enclosed in a waterproof casing. The one or more sensors can be fully enclosed within the waterproof casing. The one or more sensors can be partially enclosed within the waterproof casing and partially extending away from the casing. The system can include a filter configured to filter solids from the effluent. The one or more hardware processors can be configured to transmit the sensor data over Bluetooth. The one or more hardware processors can be configured to analyze the parameter characteristic to determine if the parameter characteristic exceeds a threshold criteria. The one or more hardware processors can be configured to transmit the alert in response to the parameter characteristic exceeding the threshold criteria. The threshold criteria can include a rate of change of the parameter over a period of time. The period of time can include one month. The one or more hardware processors can be configured to transmit the parameter characteristic to the clinician device. The monitoring device can be configured to move freely within the ostomy bag. The monitoring device can be configured to be placed without attachments in the effluent container. The monitoring device can be configured to be placed into the effluent container and to move about the effluent container. The housing can be configured to enclose a power source. The power source can include a battery or an AC power source.

The self-contained monitoring system can analyze the contents of an ostomy bag. The system can include one or more sensors configured to detect parameters associated with effluent contained in the ostomy bag; and a housing configured to enclose a wireless transmitter and the one or more sensors, the housing configured to position the one or more sensors to be in contact with the effluent, the housing configured to allow the system to be placed within the ostomy bag. The wireless transmitter can be configured to transmit sensor data to an external device. The one or more sensors can be fully enclosed in a waterproof casing. The one or more sensors can be partially enclosed within a waterproof casing and partially extending away from the casing. The system can include a filter configured to filter solids from the effluent. The system can include one or more microfluidic channels configured to transmit effluent to the at least one sensor. The system can include an agitator configured to agitate the effluent. The agitator can be configured to cause the effluent to pass through the at least one sensor. The one or more sensors can include one or more electrochemical sensors. The wireless transmitter can be configured to transmit data from the one or more sensors over Bluetooth. The system can include one or more hardware processors configured to analyze the sensor data to determine if the sensor data passes a threshold criteria. The one or more hardware processors is configured to transmit the alert in response to the sensor data passes the threshold criteria. The threshold criteria can include a rate of change of a parameter associated with the sensor data over a period of time. The period of time can include one month. The external device can include a clinician device. The self-contained monitoring system can be configured to move freely within an ostomy bag. The self-contained monitoring system can be configured to be placed without attachments in the effluent container. The self-contained monitoring system can be configured to be placed into the effluent container and to move about the effluent container. The housing can be configured to enclose a power source. The power source can include a battery or an AC power source.

For purposes of summarizing the disclosure, certain aspects, advantages, and novel features have been described herein. It is to be understood that not necessarily all such advantages can be achieved in accordance with any particular embodiment disclosed herein. Thus, the embodiments disclosed herein can be embodied or carried out in a manner that achieves or optimizes one advantage or group of advantages as taught or suggested herein without necessarily achieving others.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a block diagram of an example monitoring device for fluid waste of a user.

FIG. 1B shows an example monitoring device immersed in fluid waste and communicating with a receiver device to output readings measured by the device.

FIG. 1C shows a schematic diagram illustrating communication among the example monitoring device, a receiver device, and a remote server.

FIG. 1D shows a schematic diagram illustrating communication among an example monitoring device placed within an ostomy bag, a receiver device, and a remote server.

FIGS. 2A-2B show top perspective views of an example device for detecting biochemical parameters associated with fluid waste of a user.

FIG. 2C shows a bottom perspective view of the device of FIG. 2A.

FIG. 2D shows an exploded view of the device of FIG. 2A.

FIG. 2E shows a first portion of the device of FIG. 2A.

FIG. 2F shows a second portion of the device of FIG. 2A.

FIG. 2G shows a perspective view of an example monitoring device with a filter for fluid waste of a user.

FIG. 3A-3C illustrate views of an example assembly of an example monitoring device.

FIGS. 3D-3G illustrate views of another example monitoring device.

FIGS. 4A-4E illustrate views of electronic boards with electronic components in example monitoring devices.

FIG. 4 illustrates an example monitoring process using a device for detecting biochemical parameters associated with fluid waste of a user.

FIG. 5A illustrates schematically prior art example ostomy bags.

FIGS. 5B-5D illustrate schematic overviews of example ostomy monitoring environment according to the present disclosure.

FIG. 6 illustrates an example sensor layer of an ostomy bag.

FIGS. 7A-7B illustrate a first view of an example ostomy bag when assembled.

FIG. 7C illustrates a second view of the ostomy bag in FIG. 7A.

FIG. 7D illustrates an exploded view of the ostomy bag in FIG. 7A.

FIG. 7E illustrates a side view of the ostomy bag in FIG. 7A.

FIG. 8 illustrates an example layer of the ostomy bag in FIG. 7A.

FIG. 9 illustrates a film layer of the ostomy bag in FIG. 7A.

### DETAILED DESCRIPTION

Introduction

Systems and examples described herein relate to systems and methods for detecting long term trends in biochemical parameters associated with waste materials of a user. The waste materials can include fluid waste, solid waste, semisolid waste, and/or any combinations thereof. The waste materials can be in the form of urine and/or fecal matters. The monitoring system disclosed herein can also optionally output alerts when the long term trends fall outside a threshold and/or a threshold range. The alerts can include a warning of abnormal trends and/or likely causes of such trends. The monitoring system can be a standalone device configured to be placed within (such as free-floating within or free to move about within) or near a waste material contained in an effluent container, such as an ostomy bag, urinary catheter, toilet bowl, specimen container, or the like. The monitoring system can include a wireless transmitter, such as a Bluetooth module, for transmitting the sensor data to an external processor. The monitoring system can be used on its own and/or in combination with an ostomy system, a urine catheter, or otherwise. The monitoring system can monitor the biochemical parameters in a continuous manner and/or an intermittent manner.

The systems and methods for detecting the biochemical parameters can use a sensing device comprising a plurality of sensors, including but not limited to electrochemical sensors, optical sensors, bioMEMS sensors, acoustic sensors, and/or the like. The parameters being monitored can include, but are not limited to, concentrations of sodium, potassium, glucose, and/or the like. For example, the sensors can measure biomarkers associated with stool, such as fecal calprotectin, shigella, salmonella, campylobacter, ecoli antigen or DNA, norovirus, rotavirus, C-diff, Sodium, Potassium, Chloride, pH, glucose, lactate, lactoferrin, white cells, fecal occult blood, Fecal DNA (for example, colon cancer screening), or other markers. Additionally and/or alternatively, the sensors can measure biomarkers associated with urine, such as white cells, nitrites, blood, protein, glucose, specific gravity, osmolality, sodium, potassium, chloride, pH, lactate, E.coli, klebsiella, other urinary pathogens (for example, antigen or DNA), ammonium, phosphate, uric acid, volatile organic compounds, or other markers. The one or more sensors can also include a temperature sensor. The temperature sensor can provide information that can aid in adjusting and/or correcting sensor data for changes due to a temperature range of the fluid rather than changes of composition of the waste. The monitoring system can also include an agitation mechanism, such as a magnetic stirrer or the like, to keep a flow of the fluid in the collected waste.

The biochemical parameters measured by the sensors over time can provide long term trends which may be indicative of diseases, dehydration states, and/or others. For example, a sodium level that is lower than a predetermined range in the waste materials can be due to the user being dehydrated, and/or due to other diseases. The specific or absolute values of the biochemical parameters may not need to be calculated. The relative changes of the biochemical parameters can be monitored over a long period of time (for example, a few days, weeks, months, or longer) to obtain the long term trends of those parameters. The trend data can be shared with a clinician.

The ostomy bags used with the monitoring devices can also include one or more volumetric sensors (for example, capacitive sensors, temperature sensors, and/or others). The system can output an indication to empty and/or change the bag to a user based on, for example, detected capacitance changes in one or more capacitive sensors, which can be on the ostomy bag.

### Example Monitoring Device

As described above, a monitoring device and method for performing analysis of a sample of urine and/or fecal matters using a plurality of biosensors can include electrochemical sensor technology to detect certain biomarkers, including but not limited to Sodium, Potassium, Glucose, lactate, some combination thereof or the like and/or analyze their trends over time. The device can also monitor the pressure, temperature, and/or humidity of the samples being analyzed. The sensing device may further include a motion or position sensor, such as an accelerometer. A monitoring system utilizing the sensing device and method for collecting data can be used in any variety of urine and fecal collection vessels, including but not limited to colostomy, urosotomy, ileostomy, catheter, or other vessel. The data can be transmitted using Bluetooth or other processor to a wireless device or application.

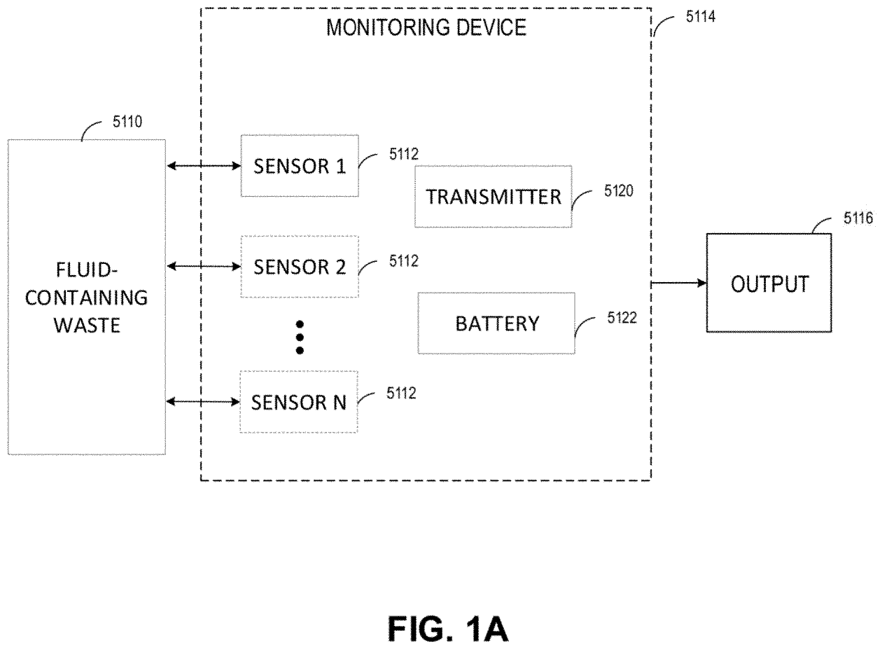

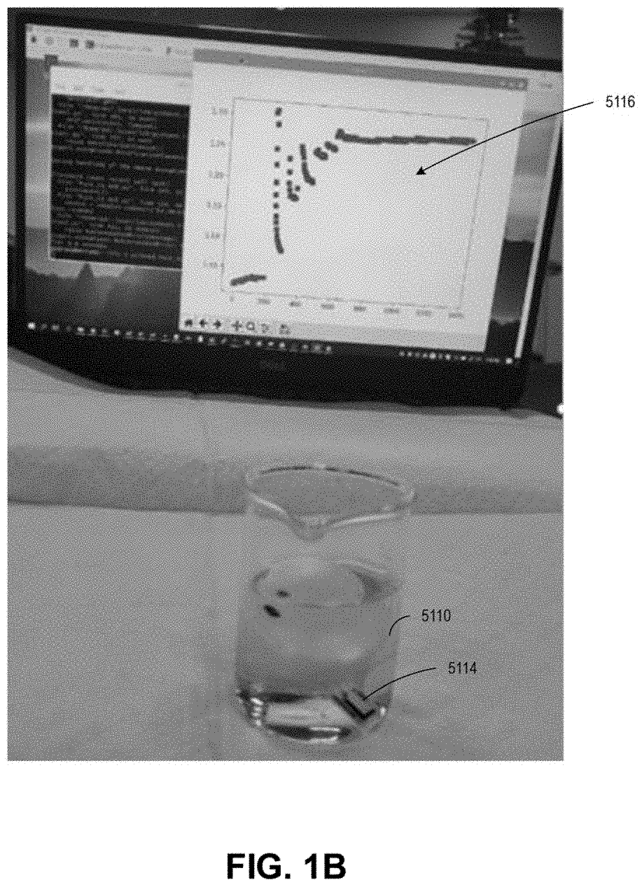

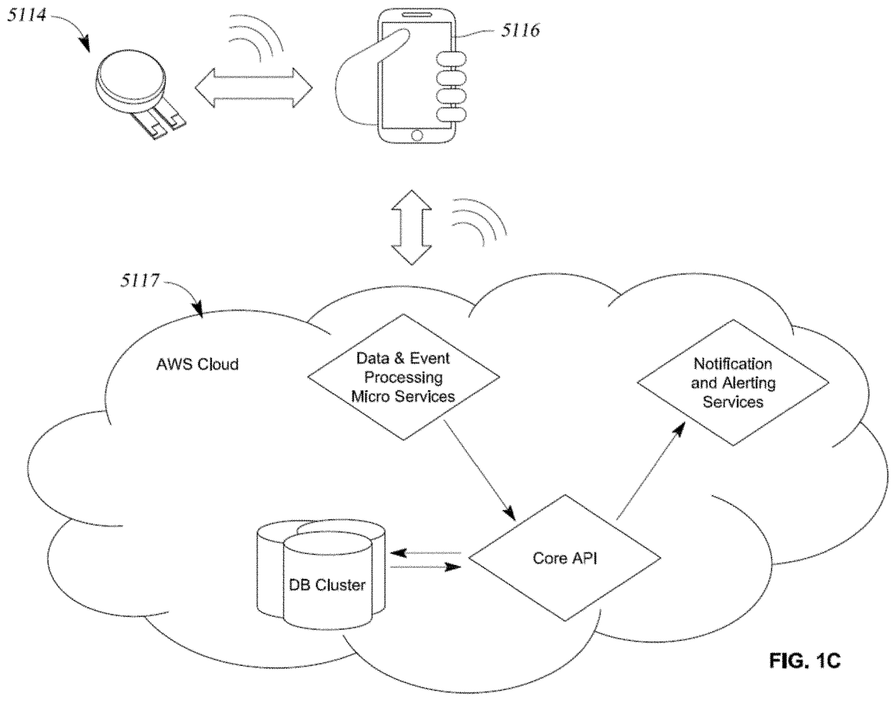

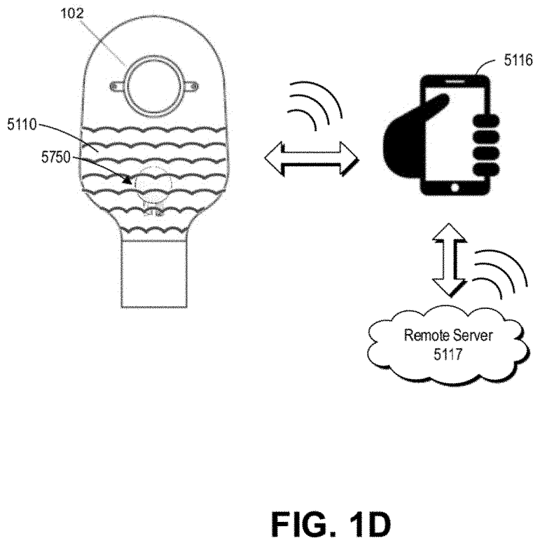

FIG. 1A shows a block diagram of an example monitoring environment of a fluid. For example, compositions of a fluid-containing waste 5110 may be measured by one or more sensors 5112 that may be part of a monitoring device 5114. The monitoring device 5114 may transmit the data measured by the sensors 5112 to an output 5116, for example, via a wireless transmitter 5120. The wireless transmitter 5120 can be a Bluetooth module or any other suitable wireless transmitter or transceiver. The data can be transmitted to the output 5116 in real time. The output 5116 can include an external server, a smartphone or tablet, or the like.

The fluid-containing waste 5110 can be any number of wastes that contains fluids. For example, the fluid-containing waste 5110 can be effluent (for example, urine and/or fecal waste), blood, serum, plasma, saliva, interstitial fluid, and/or any other fluid of a user. The fluid-containing waste 5110 can be contained in a bag, reservoir, container, catheter, other vessels, or be free flowing. For example, the fluid-containing waste 5110 can be an ostomy bag, urinary catheter, toilet bowl, specimen container, or the like.

The fluid in the fluid-containing waste 5110 can be in fluid communication with the sensor(s) 5112. The sensor(s) 5112 can be pre-calibrated to measure the analyte of interest. The sensor(s) 5112 can be individually calibrated. Alternatively or additionally, one calibration equation may apply to more than one, or all the sensors 5112. The sensor(s) 5112 can measure biochemical parameters and/or biomarkers in the fluid. The sensor(s) 5112 can be any number of sensors capable of detecting data associated with the fluid. The sensor(s) 5112 can include, but are not limited to biological, electrical, chemical, optical, temperature, ultrasonic, resistive, and/or MEMs sensors. The sensor(s) 5112 can measure markers and/or parameters in the fluid-containing waste 5110. For example, the sensor(s) 5112 can measure parameters associated with the biology and/or chemistry of the fluid-containing waste 5110. For example, the fluid-containing waste 5110 can be an effluent in an ostomy bag. The sensor(s) 5112 can measure, for example, biochemical markers for dehydration, diet, inflammation, other physiological parameters, and/or diseases, associated with the fluid-containing waste 5110. The sensor(s) 5112 can measure, directly or indirectly (such as based on an inverse relationship between sodium and potassium levels in a user's body) any number of chemical markers, including, but not limited to sodium, glucose, potassium, some combination thereof, and/or the like. For example, the sensors can measure biomarkers associated with stool, such as fecal calprotectin, shigella, salmonella, campylobacter, E.coli antigen or DNA, norovirus, rotavirus, C-diff, Sodium, Potassium, Chloride, pH, glucose, lactate, lactoferrin, white cells, fecal occult blood, Fecal DNA (for example, colon cancer screening), or other markers. Additionally and/or alternatively, the sensors can measure biomarkers associated with urine, such as white cells, nitrites, blood, protein, glucose, specific gravity, osmolality, sodium, potassium, chloride, pH, lactate, E.coli, klebsiella, other urinary pathogens (for example, antigen or DNA), ammonium, phosphate, uric acid, volatile organic compounds, or other markers.

An example of the electrochemical sensors is the ISEs. ISE includes a selective membrane or ionophore which allows the passage of the ions of primary interest (that is, the ions which are being measured) but disallows the passage of other ions. Within the ISE there is also an internal reference electrode, made of silver wire coated with silver chloride, embedded in a concentrated internal electrolyte solution which is typically used to maintain the proper functioning of the electrodes as well as enhance performance and extend the life of the electrodes. The internal electrolyte solution is saturated with silver chloride. The electrolyte solution will also contain the same ions as those that are of interest to measure.

Additionally there is a second reference electrode similar to the internal reference electrode of the ISE, but there are no ions in the internal electrolyte solution and the selective membrane is replaced by porous frit in the second reference electrode, allowing the slow passage of the internal electrolyte solution and forms a liquid junction with the external solution. The ISE and the second reference electrode are connected via a voltmeter. A measurement is made by immersing both electrodes, namely the ISE and the second reference electrode, in the same test solution.

The monitoring device can measure the amount and/or concentration of sodium, potassium, chloride, and/or glucose in the sample, and/or the pH value of the sample. The sensors in the device can detect the presence of an electrolyte within a specified range, for example, Sodium concentration greater than or equal to 0-320 mM, Potassium concentration greater than or equal to 0-200 mM, or otherwise.

The measured parameters can be used for dehydration tracking. Low sodium/potassium in the urine and/or high sodium/potassium in the stool is a cause for concern for dehydration. Measuring chloride together with sodium and potassium can provide information about the “osmolality” or concentration of the urine (another marker of dehydration). pH in the urine may be relevant in a number of conditions. Urine glucose can be a marker of high blood sugar.

The measured parameters can be applied in intraoperative monitoring during major operations. Changes in the concentration of sodium in the urine can indicate changes of blood flow to the kidneys. The concentration of sodium in the urine decreases when the blood flow is reduced.

Sodium in the urine can additionally or alternatively be tracked as a marker of sodium intake, which is a risk factor for high blood pressure. The monitoring device 5114 can be available for helping people track and self-manage their blood pressure risk. There are various other benefits of quantifying urinary sodium for respiratory, endocrine, and/or liver conditions.

The monitoring device 5114 can alternatively or additionally detect more complex molecules, such as fecal calprotectin (a measure of activity of inflammatory bowel disease and measured from the stool) and/or markers of infection such as c. difficile.

Whilst urine is a pure liquid medium, fecal effluent has the presence of both liquid and solid phases. A filter can optionally be placed between the sensor(s) 5112 and the fluid-containing waste 5110 so that only the liquid in the waste makes contact with the sensor(s) 5112. The filter can protect the sensor(s) 5112 from solids that may be present in the fluid-containing waste 5110 and may interfere with the sensing and/or damage the sensors. Additionally or alternatively, the sensor(s) 5112 may not be in direct contact with a fluid-containing waste 5110 source, reservoir, container, or vessel. For example, fluid-containing waste 5110 may be delivered or transmitted to the sensor(s) 5112. For example, the fluid-containing waste 5110 may be delivered by one or more fluid channels. The one or more fluid channels may be macro-, micro-, or nano-fluidic channels. Advantageously, the use of fluid channels can protect the sensor(s) 5112 from solids in the fluid-containing waste 5110 and allow the transportation of the liquid to the sensitized electrode area of the device 5114, and can reduce the need for the rest of the device to be watertight. A wicking membrane or device(s) may also be implemented to direct the flow of the liquid straight to the sensitized electrode area.

The fluid can be mixed or agitated prior to, during, and/or after being delivered or transmitted to, from, and/or within the sensor(s) 5112. Additionally or alternatively, the agitation of the fluid can serve to pass the fluid to, from, within, and/or through the sensor(s) 5112. Advantageously, the agitation of the fluid-containing waste 5110 can improve the accuracy of the sensor(s) 5112 by keeping the fluid-containing waste 5110 in motion. A sensor 5112 in static solution (as shown in FIG. 1B) may result in a signal dropping off over time. Thus, a moving solution can help to improve the detection of the signal. The fluid can be agitated with any suitable agitation mechanism. For example, the fluid can be agitated with a sonic or mechanical mixer.

The sensor(s) 5112 can be part of a monitoring device 5114. The sensor(s) 5112 can be partially or completely enclosed within a housing of the monitoring device 5114. The housing can be generally cylindrical. The housing can have a diameter of about 20 mm to about 40 mm, or about 23 mm to about 37 mm, or about 26 mm to about 33 mm, or about 30 mm. The housing can have a height of about 4 mm to about 16 mm, or about 6 mm to about 14 mm, or about 8 mm to about 12 mm, or about 10 mm. Alternatively, the housing can have other suitable shapes. The housing can be formed from a robust material and fully (or at least partially) sealed in order to withstand immersion in a urine or faecal effluent sample. The material of the housing does not allow penetration of moisture inside an internal sealed chamber as moisture may damage the embedded electronics and sensors. Examples of the housing material can include hard plastics, for example, Polyester, LDPE, HDPE, PET, PVC and Polycarbonate.

The device 5114 can monitor a user's fluid continuously, intermittently, upon demand by a user, clinician, and/or a controller, and/or any combination thereof. The monitoring device 5114 can include one or more sensor(s) 5112. For example, the monitoring device 5114 can include four or eight sensor(s) 5112, or any other number of sensors. The one or more sensor(s) 5112 can be the same or different sensors. For example, one or more of the sensor(s) 5112 can measure the same or similar data associated with the fluid-containing waste 5110 so as to provide redundancy. Additionally or alternatively, the sensor(s) 5112 can be different sensors. For example, the sensor(s) 5112 can be sensors capable of measuring different parameters and/or data associated with the fluid-containing waste 5110. For example, the sensors 5112 can include one or more of a temperature sensor, a resistive sensor, an ultrasonic sensor, an electrochemical sensor, and/or an accelerometer. The temperature sensor may be capable of measuring the temperature of the fluid-containing waste 5110. The temperature sensor may be used to correlate the sensor signals to the temperature of the fluid. The correlation can allow for determining portions of the signal change that is due to temperature changes instead of composition changes in the fluid. The resistive sensor may be capable of measuring the viscosity of the fluid-containing waste 5110. The ultrasonic sensor may be capable of measuring the volume of the fluid, for example, in an ostomy bag. The electrochemical sensor may be capable of measuring chemical content of the fluid-containing waste 5110. The accelerometer may be capable of measuring a posture and/or movements of a user. Any of the sensors in this example can be replaced by other suitable sensors disclosed herein.

Because the monitoring device 5114 is designed to be versatile and robust in hostile environments, the monitoring device 5114 can be placed in any number of locations in relation to a fluid-containing waste 5110. For example, the monitoring device 5114 can be placed on, within, or adjacent to an ostomy bag (such as an example monitoring device 5750 placed within an ostomy bag 102 as shown in FIG. 1D) or wafer, on, within, or adjacent to a catheter, and/or can be a stand-alone device. Fluidics can be used to transmit fluids in the fluid-containing waste 5110 to the sensor(s) 5112 of the monitoring device 5114. For example, the monitoring device 5114 may be disposed on an ostomy wafer. Fluidic channels can carry some portion of the contents of the ostomy bag to the monitoring device 5114 disposed on the ostomy wafer. Additionally or alternatively, the monitoring device 5114 may be placed on or adjacent to the fluid-containing waste 5110 contained in a fluid holding vessel, such as an ostomy bag. For example, the monitoring device may be placed directly adjacent to the effluent content of an ostomy bag such that the sensor(s) 5112 can detect parameters of the effluent without the need for fluidic transport. The device 5114 can also be deployed directly into a catheter bag or be placed in a dedicated sensing collection cup, suspended in a working lavatory or integrated with/into medical devices, such as a urine collector. Additionally or alternatively, the monitoring device 5114 can be a stand-alone device without a direct connection to a fluid-containing waste 5110 or to a vessel containing a fluid-containing waste 5110. For example, the monitoring device 5114 can be capable of receiving a fluid-containing waste 5110 to measure by the sensor(s) 5112 and/or capable of being placed directly in contact with a fluid-containing waste 5110 without being disposed onto something. As shown in FIG. 1B, the fluid-containing waste 5110 (for example, a urine sample) can be contained in a beaker or another container, with the monitoring device 5114 fully immersed in the waste 5110. Data output 5116 can be wirelessly transmitted from the monitoring device 5114 to a laptop and optionally displayed in real time.

The monitoring device 5114 can include any number of electronic components. For example, the monitoring device 5114 can include any combination of processing electronics, one or more hardware processors, one or more batteries, a wireless transmitter 5120, and/or other electrical components. The monitoring device 5114 can process the data from the sensor(s) 5112 using the electronic components. For example, monitoring device 5114 can pre-process data from the sensor(s) 5112 to, for example, remove noise (electrical and/or mechanical) in the data using a filter, smoothing and/or noise removal algorithms, and/or otherwise, or amplify a signal from the sensor(s) 5112.

The monitoring device 5114 can wirelessly (preferably) or with wires transmit data from the sensor(s) 5112 to an output 5116. As shown in FIGS. 1C-1D, the output 5116 can include a mobile device, application, display, or other means of storing, analyzing, and/or displaying data. For example, the output 5116 can be a clinician device or a user device. An integrated BLE (Bluetooth Low Energy) chip can communicate the real time analytical data from the device 5114 to the mobile device 5116 or another receiver device. A mobile application available on iOS and Android platforms (or other suitable platforms), can receive the transmitted data and optionally display the transmitted data. A user may login to the application to review the transmitted data. As shown in FIG. 1C, data received at the receiver device can further be pushed to a remote server, for example, the Amazon Web Services (AWS) cloud service 5117 or otherwise, and downloaded to other dashboards and platforms alike, for example, for physicians access. The data can also optionally be stored in a database (DB) cluster on the cloud service 5117. Additionally, the cloud service 5117 can process the data so as to provide notification and alerting services (for example, for physician notification and alerting). As shown in FIG. 1D, the receiver device 5116 can be connected to any remote server 5117.

The monitoring device 5114 can transmit data on demand, periodically, automatically, semi-automatically, or in response to a condition. The monitoring device 5114 can selectively transmit data to an output 5116 based on the content of the data. For example, the monitoring device 5114 can analyze the data to determine a long term trend. If the long term trend is deemed of interest, the monitoring device 5114 can transmit the data to an output 5116. Additionally or alternatively, the monitoring device 5114 can collect and transmit data (which can be preprocessed data), which can be analyzed by the recipient of the data, such as the clinician device or the user's device. Additionally or alternatively, the monitoring device 5114 can transmit data in real time or periodically to the output 5116. For example, the monitoring device 5114 can periodically transmit data from the sensor(s) 5112 to an output 5116, such as a user device. The periodic transmission of data can allow for backup storage of the sensor data and/or can alert a user to parameters associated with the fluid-containing waste 5110. For example, the monitoring device 5114 may measure the effluent in an ostomy bag of a user. The monitoring device 5114 may transmit periodic or real time data to the user's mobile device, allowing the user to make better informed decisions relating to their ostomy bag use.

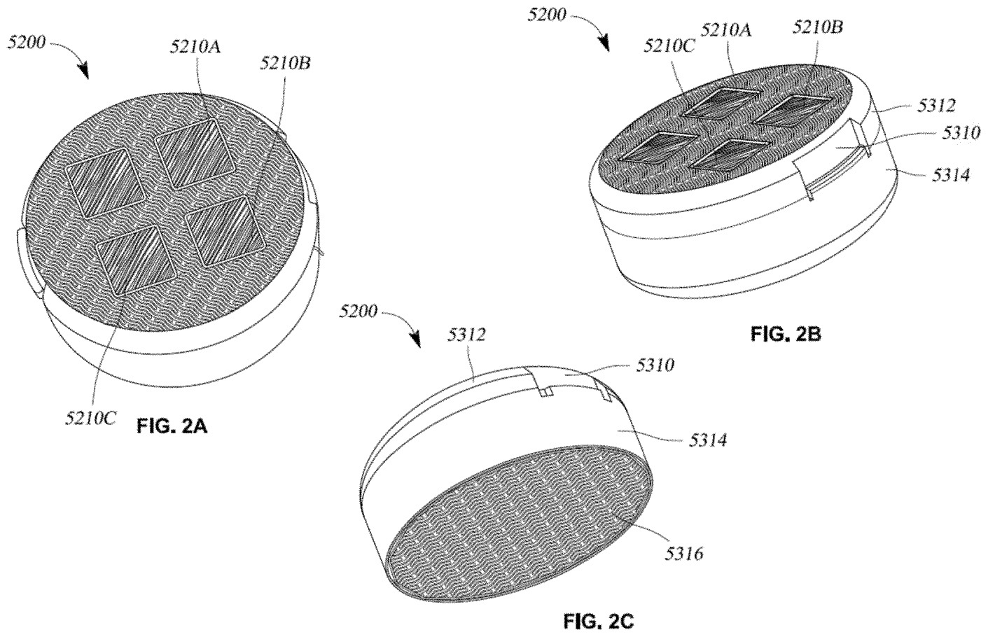

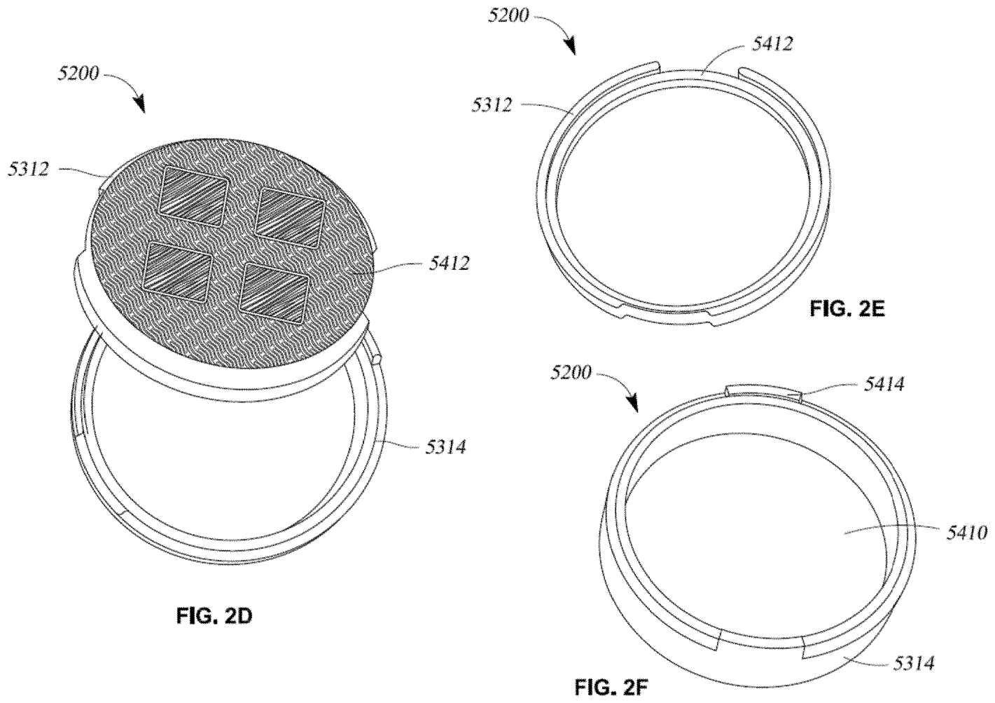

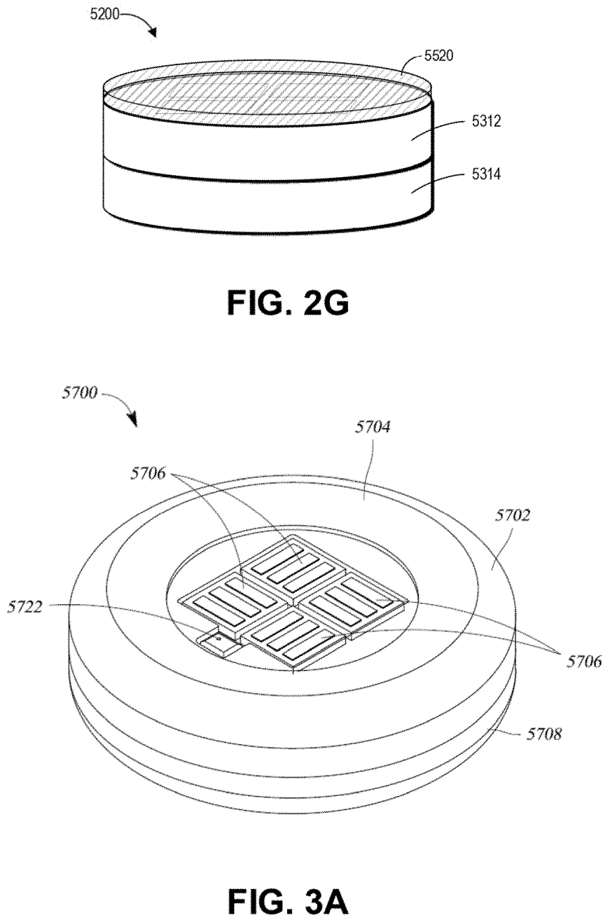

FIGS. 2A-2F show perspective views of an example device 5200 for detecting parameters associated with a fluid, such as an effluent in an ostomy bag or urine in a catheter.

The device 5200 can include one or more wells or cavities in which sensors for measuring parameters associated with a fluid can be disposed. For example, as shown in FIG. 2A, the device 5200 can include four, eight, or other number of cavities (5210A, 5210B, 5210C, 5210D). The cavities can be of any number of sizes, shapes, or depths to accommodate any number of sensors of different sizes and shapes. The sensors can be any number or type of sensor. The sensors may be permanently or removably affixed within the cavities. The cavities may include electrical connections by which the sensors disposed in the cavities can electrically connect and/or communicate with electronic components that may be disposed within the device 5200. For example, a temperature sensor disposed in a cavity in device 5200 may make electrical contact with leads adjacent to or within the device cavity to connect to a hardware processor within the interior of the device 5200.

As shown in FIGS. 2B and 2C, the device 5200 can have a top portion 5312 and a bottom portion 5314. The top portion 5312 and the bottom portion 5314 can couple to form a watertight or water-resistant seal. The top portion 5312 and the bottom portion 5314 can couple through a locking, sealing, latching, clipping, other closing mechanism 5310, or some combination thereof. For example, as illustrated in FIGS. 2B and 2C, the closing mechanism 5310 can include one or more clips for holding the portions together. Additionally or alternatively, the closing mechanism 5310 can include screws for holding the portions together.

As shown in FIGS. 2D, 2E, and 2F, the device 5200 can be opened to access an internal compartment 5410 formed between the top portion 5312 and the bottom portion 5314. The internal compartment 5410 can receive any number of electronic components. For example, the internal compartment 5410 can include a power source component, one or more processors, pre-processing electronics, one or more transmitters, and/or other electrical components. The internal compartment 5410 can optionally be compartmentalized to better secure the electronics and/or other components placed within the internal compartment 5410.

The internal compartment 5410 may be accessible by a user. For example, the closing mechanism 5310 may be operable such that a use can expose and seal the internal compartment 5410. The user may be able to remove and replace electrical components in the internal compartment 5410. For example, the user may be able to remove, replace, charge, or otherwise access a battery or battery components within the device 5200 by accessing the internal compartment 5410.

The device 5200 can include one or more power sources or power source components, such as a battery 5122. The power source components may be disposed in the internal compartment 5410. The power source component may be capable of providing power to the sensors and other electrical components that may be part of or disposed on and within the device 5410. The power source may have a set charge. For example, the power source may have enough charge to power the electrical components on, within, or part of the device for a number of hours or days. For example, the power source may have power for 3 to 4 days, or any other period of time of continuous use. Optionally, the power source can include an AC power source.

The device 5200 can include a transmitter component. The transmitter component may be disposed in the internal compartment 5410. The transmitter component may be a wireless transmitter 5120. The transmitter component can transmit sensor and/or hardware data to an external device. The transmitter component can include any number of transmission technologies. For example, the transmitter component can be a Bluetooth, radio frequency, or other wireless transmitter 5120. For example, the device 5200 can include a Bluetooth transmitter. The Bluetooth transmitter can transmit sensor data to a user mobile device.

The device 5200 can include processing electronics for processing one or more signals from the sensors. The processing electronics may be disposed in the internal compartment 5410. The processing electronics can be configured to analyze data obtained by the one or more sensors or prepare data from the sensor(s) for analysis and storage. For example, the processing electronics can include one or more filters to improve a signal from the sensor(s) and/or reduce noise, including but not limited to electrical noise, mechanical noise. The pre-processing electronics can include some combination of electronics including, but not limited to: an analog to digital converter (ADC), an anti-aliasing filter (AAF), and/or operational amplifier (op-amp). The operational amplifier (op-amp) can increase the amplitude, as well as transform the signal, such as from a current to a voltage. An anti-aliasing filter (AAF) can then process the output signal from the op-amp to restrict a bandwidth of the output signal from the op-amp to approximately or completely satisfy the sampling theorem over a band of interest. An analog-to-digital convertor (ADC) can convert the output signal from the AAF from analog to digital. The output signal from the ADC can then be sampled by a processor at a relatively high speed. The result of the sampling can next be downsampled before waveform analysis may be performed.

Additionally or alternatively, the device 5200 can make use of one or more noise reduction algorithms. For example, the device 5200 may implement a noise reduction algorithm by one or more hardware processors that can be disposed in the internal compartment 5410.

The device 5200 can include one or more hardware processors. One or more hardware processors may be disposed in the internal compartment 5410. The one or more hardware processors may execute one or more programs for determining parameters from the sensor data. For example, the one or more hardware processors may analyze data obtained from the one or more sensors. The data analysis can include analyzing signals from the one or more sensors to determine one or more physiological parameters or biomarkers associated with the fluid. Additionally or alternatively, the one or more hardware processors may analyze the signals or determined parameters or biomarkers for trends or other analysis.

FIG. 2G shows a perspective view of the example device 5200 with a solid waste filter 5520. A solid waste filter 5520 can be placed between the top portion 5312 and a fluid-containing waste to protect the sensors that may be disposed in the top portion 5312 from solids that may be present in the fluid. Additionally or alternatively, the fluid may be delivered or transmitted to the sensor. For example, the fluid-containing waste 5110 may be delivered by one or more fluid channels. The one or more fluid channels may be macro-, micro-, or nano-fluidic channels. Advantageously, the use of fluid channels can protect the sensor(s) from solids in the fluid and can reduce the need for the rest of the device to be watertight.

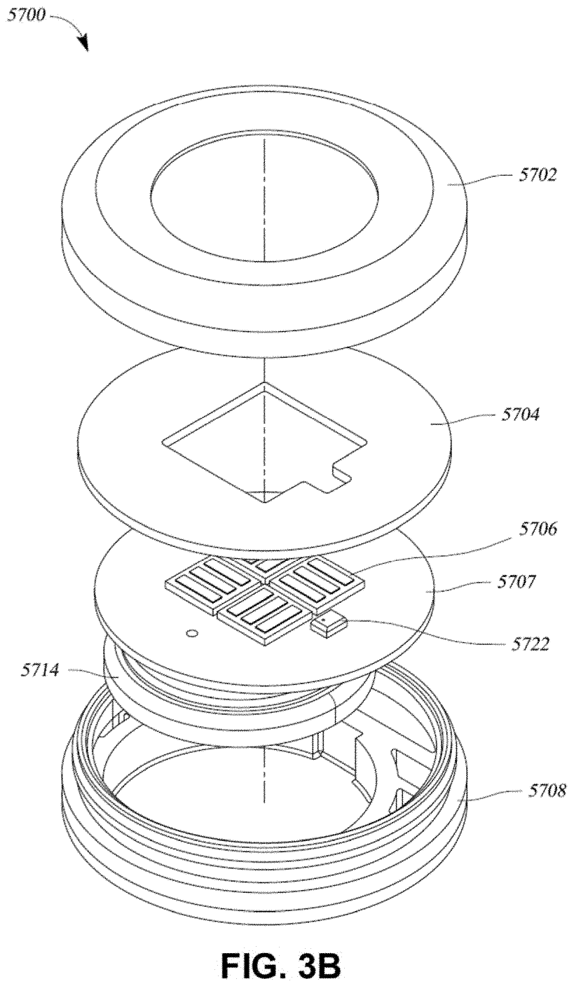

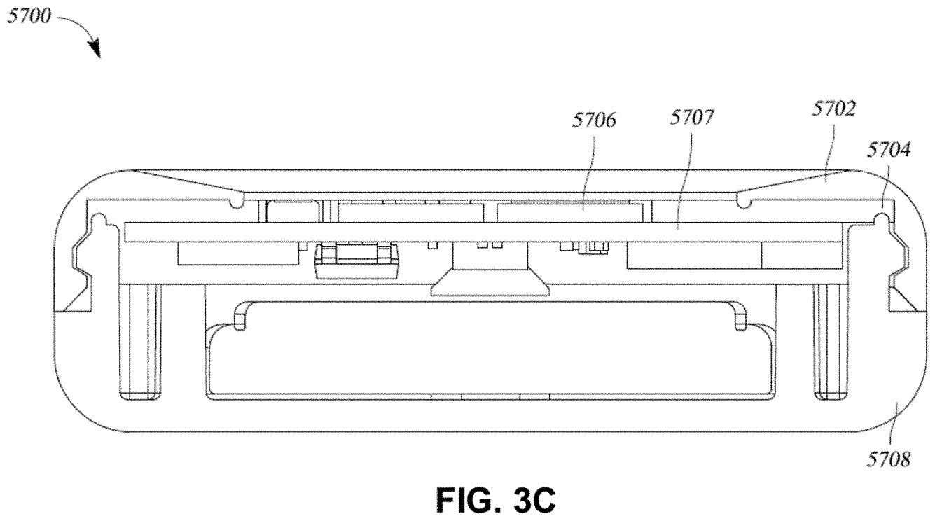

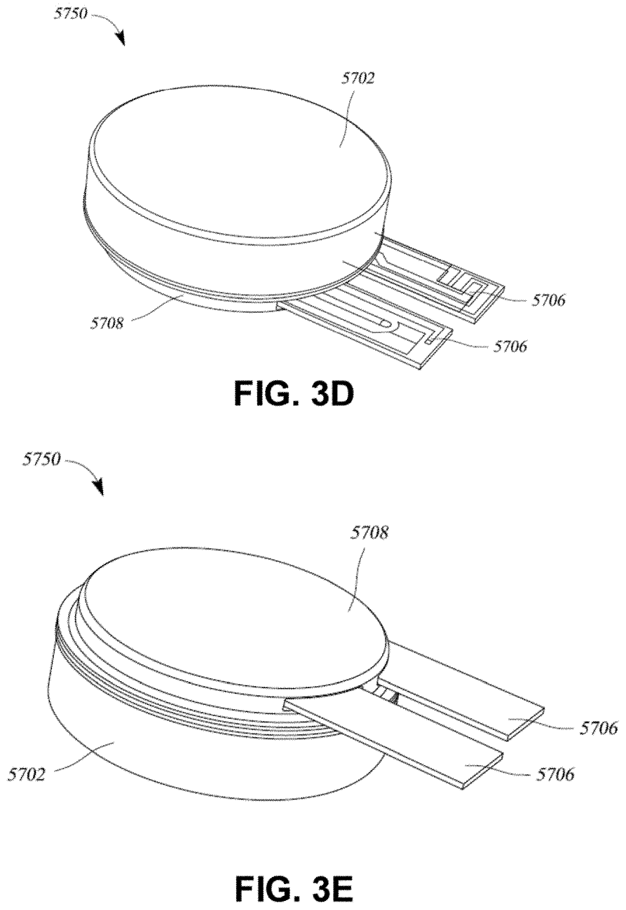

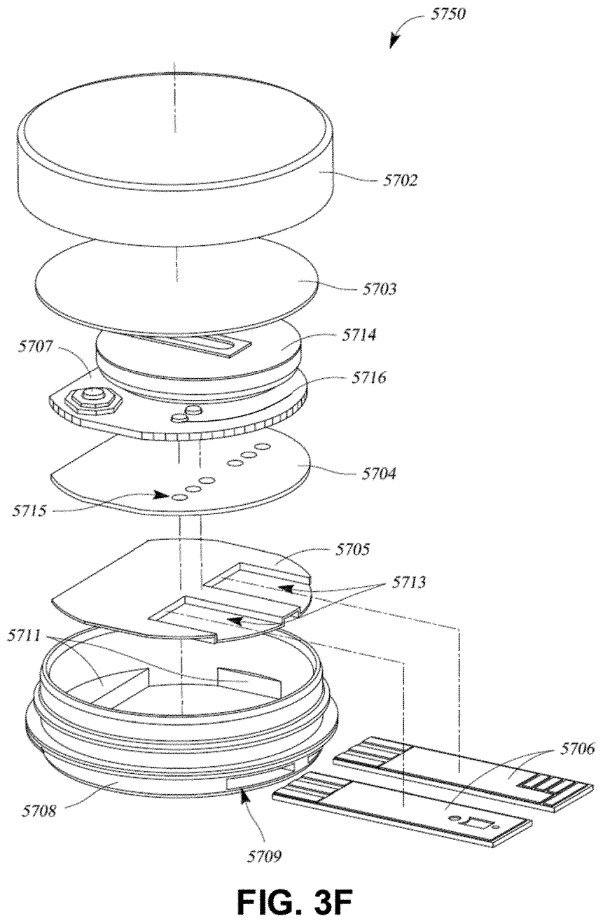

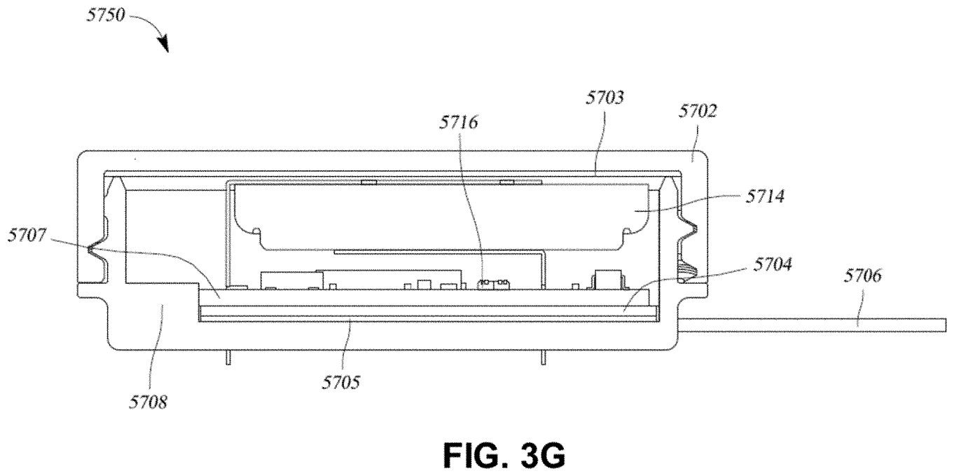

FIG. 3A illustrates an assembled view of an example device 5700. Features of the device 5700 and features of the device 5200 can be incorporated into each other. FIG. 3B illustrates an exploded view of an example device 5700 of FIG. 3A. FIG. 3C illustrates a cross sectional view of the device 5700 of FIG. 3A. The example device 5700 can include a housing, a sealing component 5704, and electronics, for example, one or more sensors 5706. The housing can include a top casing 5702 and a bottom casing 5708.

The top casing 5702 can include a water resistant material with one or more openings. The one or more openings can be configured to allow exposure of one or more other components of the device 5700, such as the sensors 5706, to a fluid being measured. For example, a device 5700 can include one or more sensors 5706 (for example, eight sensors) and the top casing 5702 can include a substantially central opening in which the sensor(s) can be exposed. A fluid or moisture barrier 5704 can surround the sensors and prevents fluid from entering a chamber enclosed by the housing via the opening of the top casing 5702.

The bottom casing 5708 can include a water resistant material with one or more inner compartments. The one or more inner compartments can be configured to accommodate and/or retain one or more internal components of the device 5700, such as an electronics board 5707 with the sensors 5706. The top casing 5702 and bottom casing 5708 may be configured to mate. The mating of the top and bottom casings 5702, 5708 can form a watertight side wall of the housing. For example, the casings 5702, 5708 may snap, screw, bond using adhesives, or otherwise be coupled to form a moisture resistant seal. The casings 5702, 5708 may be configured to be reversibly coupled such that a user can replace interior components of the device 5700, such as the electronics board 5707, or other components (for example, a battery 5714 or one or more sensors).

The fluid or moisture barrier 5704 can include a sealing component. The barrier 5704 can be made of any number of materials, technologies, or components capable of providing moisture protection to one or more electronic components of the device 5700. For example, the fluid or moisture barrier 5704 can include a gasket. The gasket may be configured to include an opening for the one or more sensors 5706 so that the one or more sensors can be exposed to a fluid being measured, while creating a barrier between the fluid being measured or other source of moisture and internal components of the device 5700, such as one or more hardware processors or batteries. The gasket may be compressed between one or more component layers of the device 5700 to seal any gaps through which fluid or moisture can enter the housing. For example, the gasket may be compressed between the top casing 5702 and the electronics board 5707 or bottom casing 5708 when the top and bottom casings 5702, 5708 are coupled together. The gasket may be of any suitable fluid or moisture resistant material, such as silicone, rubber, or other material.

The housing can accommodate any number of electronic components associated with the device 5700. As illustrated in FIG. 3C, the electronic components can include one or more sensors 5706, a circuit board 5707, a battery 5714, and other electronics for powering the device 5700 or measuring, transmitting, or processing data, such as the electronic components discussed herein.

FIGS. 3D-3E illustrates an assembled view of an example device 5750. Features of the device 5750, features of the device 5700, and features of the device 5200 can be incorporated into one another. FIG. 3F illustrates an exploded view of an example device 5750 of FIG. 3D. FIG. 3G illustrates a cross sectional view of the device 5750 of FIG. 3D. The example device 5750 can include a housing, a plurality of sealing components 5703, 5704, 5705, and electronics, for example, a plurality of sensors 5706 and an electronics board 5707. The housing can include a top casing 5702 and a bottom casing 5708.

The top casing 5702 and the bottom casing 5708 can each include a water-resistant material. An inner chamber can be formed by mating the top casing 5702 with the bottom casing 5708. The top and bottom casings 5702, 5708 may snap, screw, bond using adhesives, or otherwise be coupled to form a moisture resistant seal. The casings 5702, 5708 may be configured to be reversibly coupled such that a user can replace interior components of the device 5750, such as the electronics board 5707, or other components (for example, the battery 5714 or any of the sealing components).

The inner chamber can be water-tight by further including the plurality of sealing components, for example, a first sealing component 5703 and a second sealing component 5704. The inner chamber can enclose the electronics board 5707 and the battery 5714. The electronics board 5707 and the battery 5714 can be sandwiched between the first and second sealing components 5703, 5704 when the device 5750 is fully assembled. An inner side wall of the bottom asing 5708 may include one or more protrusions or steps 5711 so that a well in the bottom casing 5708 can have a shape conforming substantially to an outer shape of the electronics board 5707. The first sealing component 5703 having an outer diameter that is substantially the same or slightly bigger than an inner diameter of the top casing 5702. The second sealing component 5704 can have an outer shape that substantially matches the shape of the well in the bottom casing 5708 and/or the outer shape of the electronics board 5707.

The sensors 5706 are not mounted onto the electronics board 5707 in the device 5750, but are still in electrical communication with the electronics board 5707. The bottom casing 5708 can include a plurality (for example, two for more) openings 5709 each configured to receive one of the sensors 5706. When assembled, the sensors 5706 are partially received within the housing and partially extending away from the housing. The sensors 5706 can be ion-selective, for example, include a sodium sensor and a potassium sensor.

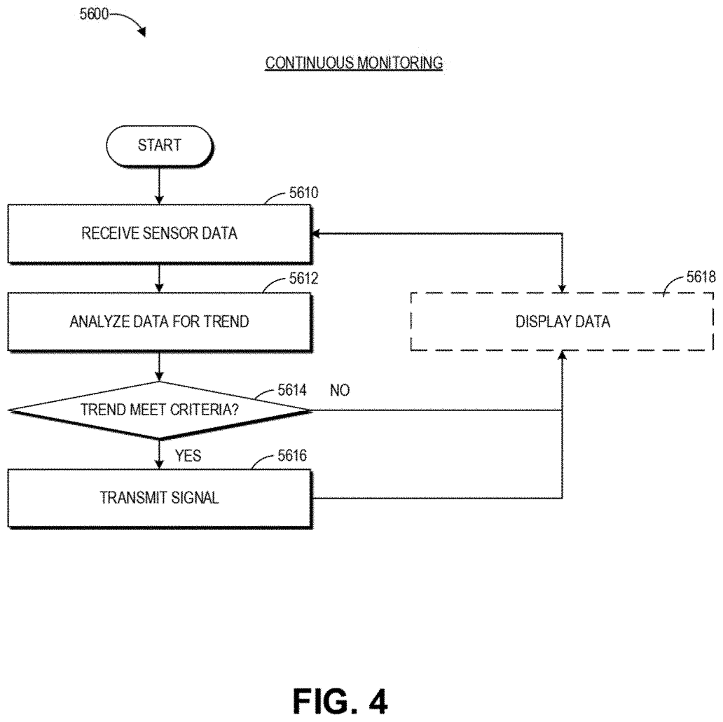

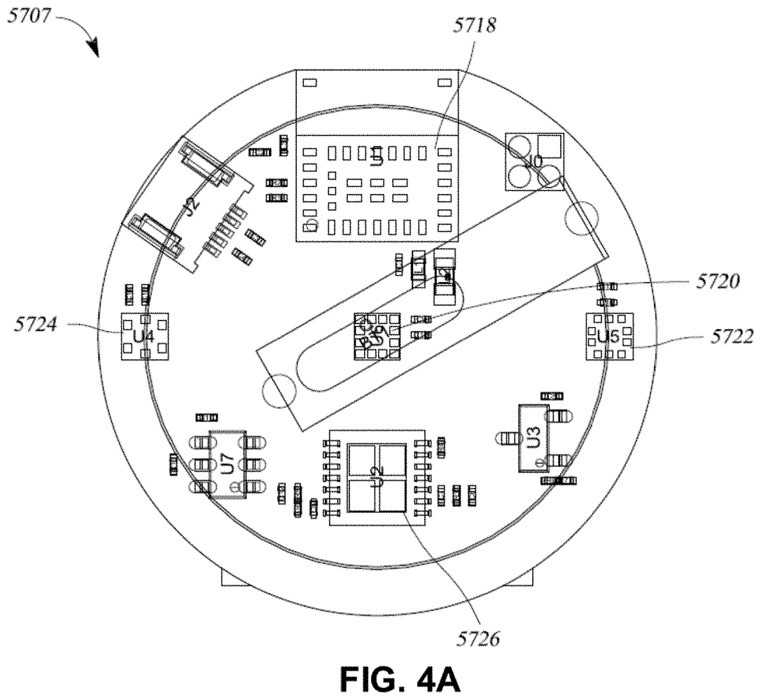

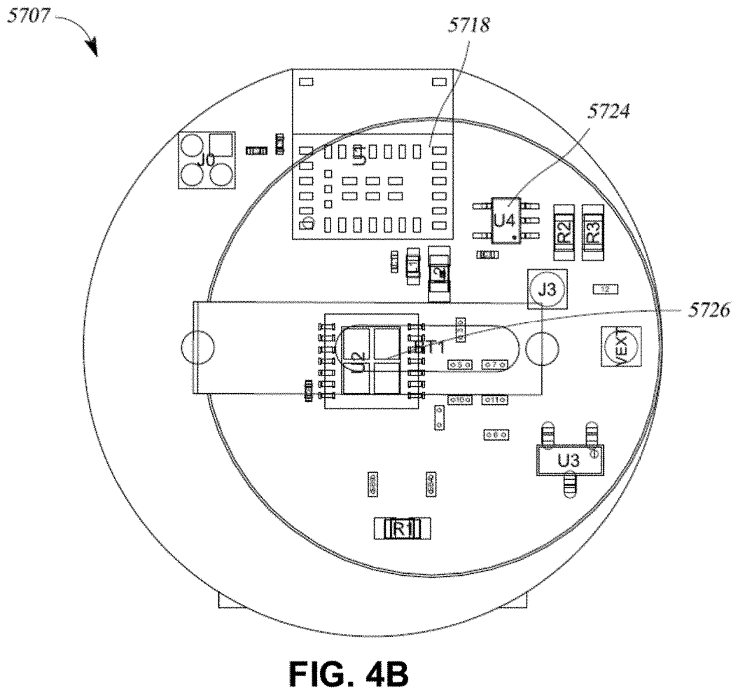

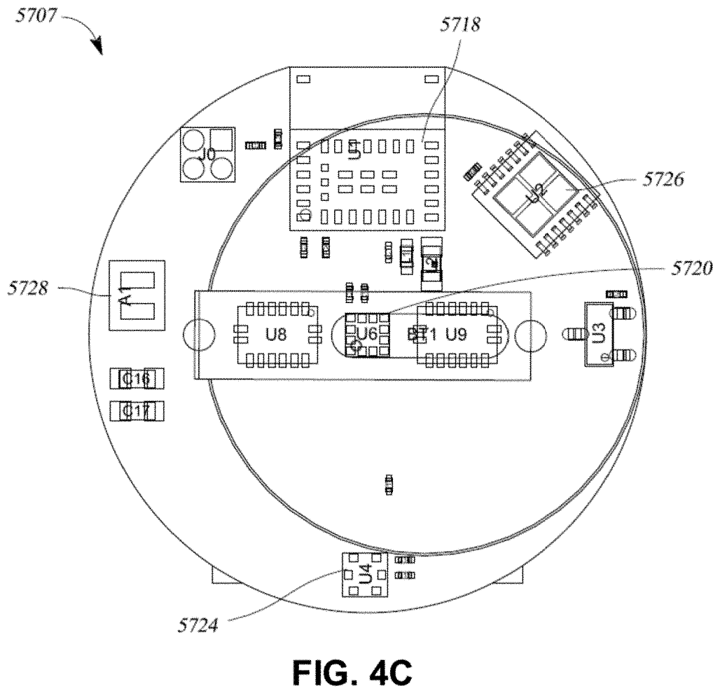

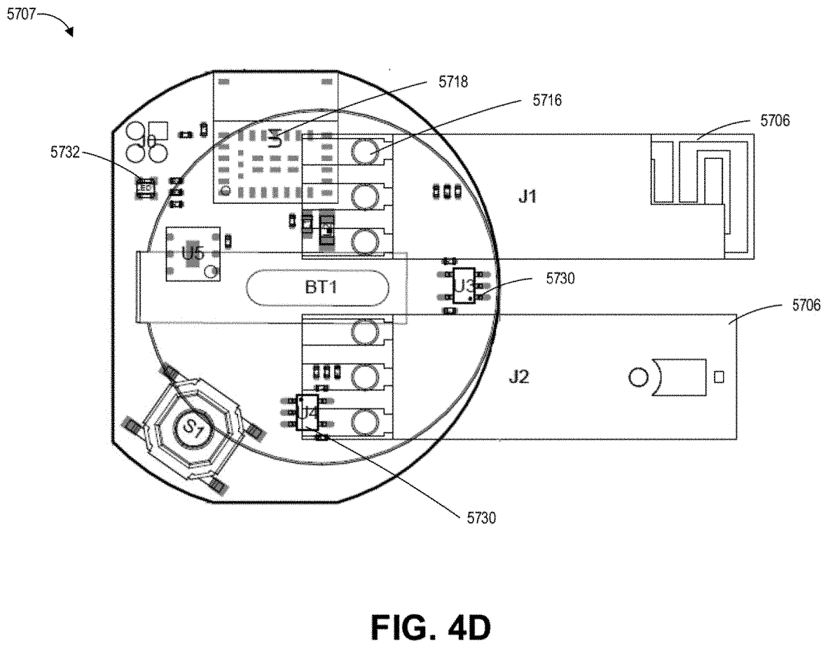

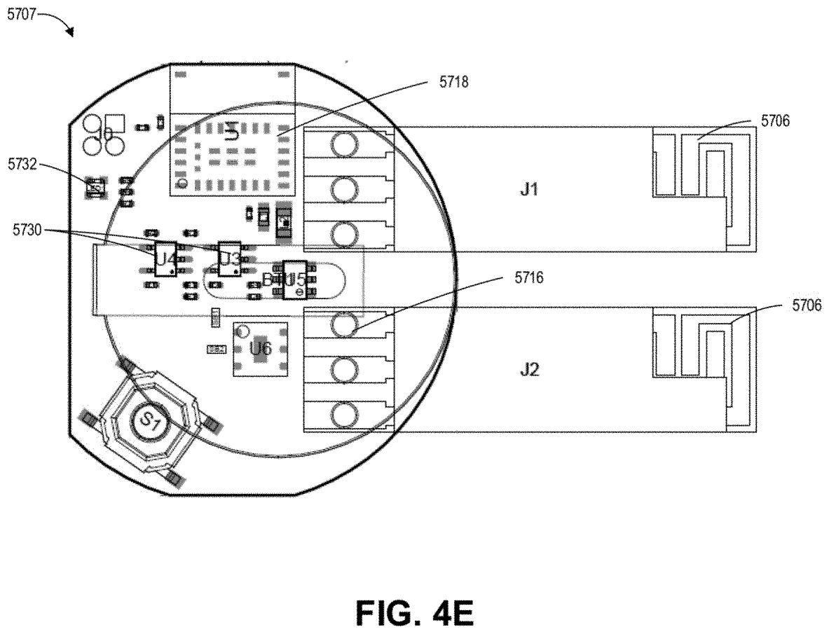

The sensors 5706 may not be received within the watertight internal chamber. Portions of the sensors 5706 inside the housing can be sandwiched between the second sealing component 5704 and a third sealing component 5705. The third sealing component 5705 can have an outer shape that substantially matches the shape of the well in the bottom casing 5708 and/or the outer shape of the electronics board 5707. The third sealing component 5705 can have grooves 5713 each configured to snugly fit the portion of the sensor 5706 inside the housing. The second sealing component 5704 can include a plurality of openings 5715 configured to allow electrical connectors 5716 (see also FIGS. 4D-4E) from the electronics board 5707 to form electrical connections with the sensors 5706. The openings 5715 can form a watertight seal around the electrical connectors 5716. The second and third sealing components 5704, 5705 can prevent water from entering the housing via the openings 5709. Optionally, only the portions of the sensors 5706 extending outside the housing can be exposed to the testing sample.

The first, second, and third sealing components 5703, 5704, 5705 can be made of any number of materials, technologies, or components capable of providing moisture protection to one or more electronic components of the device 5750. The first, second, and third sealing components 5703, 5704, 5705 can include a gasket. The gasket may be compressed between one or more component layers of the device 5750 to seal any gaps through which fluid or moisture can enter the inner chamber of the housing. For example, the gasket 5703 may be compressed between the top casing 5702 and the electronics board 5707, the gaskets 5704, 5705 may be compressed between the electronics board 5707 and bottom casing 5708 when the top and bottom casings 5702, 5708 are coupled together. The gasket may be of any suitable fluid or moisture resistant material, such as silicone, rubber, or other material.

The housing can accommodate (fully, such as being mounted on the electronics board 5707, or partially) any number of electronic components associated with the device 5750. As illustrated in FIG. 3G, the electronic components can include one or more sensors 5706, the circuit board 5707, the battery 5714, and other electronics for powering the device 5750 or measuring, transmitting, or processing data, such as the electronic components discussed herein.

Details of the electronic board 5707 will now be described with reference to FIGS. 4A-4E. The layout and/or combinations of the electronic components on the electronic board 5707 shown in FIGS. 4A-4E are for illustrative purposes and are not limiting, and can be implemented in any of the monitoring device examples disclosed herein. The monitoring device can include any number of the sensors disclosed herein in any combination.

All the electronics board examples 5707 can include a wireless communication module, for example, a Bluetooth module 5718. FIG. 4A illustrates an electronics board 5707 including an accelerometer 5720, a pressure sensor 5722, a humidity/temperature sensor 5724, and a potentiostat 5726 for glucose detection. FIG. 4A illustrates an electronics board 5707 including an accelerometer 5720, a pressure sensor 5722, a humidity/temperature sensor 5724, and a potentiostat 5726 for glucose detection. FIG. 4B illustrates an electronics board 5707 including a humidity/temperature sensor 5724 and a potentiostat 5726 for glucose detection. The electronics boards 5707 in FIGS. 4A-4B can support detection of sodium, potassium, and the pH of the testing sample. FIG. 4C illustrates an electronics board 5707 including up to four ZP 5×5 sensors (see, e.g., the sensors 5706 shown in FIG. 3A) manufactured by Zimmer and Peacock (Napa, Calif.). The ZP sensors can function as an accelerometer 5720, a pressure sensor 5722 (located on the opposite side of the electronics board 5707 with the ZP sensors 5706 as shown in FIGS. 3A-3B), a humidity/temperature sensor 5724, and a potentiostat 5726 for glucose detection. The electronics board 5707 in FIG. 4C can also include an NFC antenna 5728. FIGS. 4D-4E illustrate electronics boards 5707 including operational amplifiers 5730 coupled to the ion-selective (sodium and potassium) sensors 5706 such as shown in FIGS. 3D-3E. The electronics boards 5707 in FIGS. 4D-4E can also include an LED 5732 for user feedback. The LED 5732 can include an RGB LED (that is, Red, Green, and Blue). The LED 5732 can provide user feedback in a variety of ways, for example, by notifying a user whether the device is connected to the receiver device, whether the readings are within a predetermined threshold or limit, or otherwise.

FIG. 4 illustrates an example monitoring process 5600. The monitoring process may use data received from a monitoring device 5114 or device 5200 and/or be implemented by one or more hardware processors operating as part of or in conjunction with a monitoring device 5114 or device 5200. The process can be performed, for example, by a processor on a clinician device (such as a computer) or a user's device (such as a smartphone or tablet). For example, as illustrated in FIG. 4, the monitoring process 5600 can include a data receiving step at block 5610, a data analysis step at block 5612, a criteria analysis at block 5614, and a signal transmission step at a block 5616.

At a block 5610, the process 5600 can include receiving data from sensors. The sensors may be sensors 5112 that are part of a monitoring device 5114 as illustrated in FIGS. 1A-1D. The sensors may detect data associated with a fluid. The fluid can be any fluid. For example, the fluid may be a fluid-containing waste 5110 as illustrated in FIGS. 1A-1D. The sensors can measure markers and/or parameters in the fluid. For example, the sensors can measure parameters associated with the biology and/or chemistry of the fluid. For example, the fluid can be an effluent in an ostomy bag. The sensors can measure biochemical inflammatory markers associated with the effluent. The sensors can measure any number of chemical markers, including, but not limited to sodium, glucose, potassium, some combination thereof, and/or the like. For example, the sensors can measure biomarkers associated with stool, such as fecal calprotectin, shigella, salmonella, campylobacter, E.coli antigen or DNA, norovirus, rotavirus, C-diff, Sodium, Potassium, Chloride, pH, glucose, lactate, lactoferrin, white cells, fecal occult blood, Fecal DNA (for example, colon cancer screening), or other markers. Additionally and/or alternatively, the sensors can measure biomarkers associated with urine, such as white cells, nitrites, blood, protein, glucose, sp0ecific gravity, osmolality, sodium, potassium, chloride, pH, lactate, E.coli, klebsiella, other urinary pathogens (for example, antigen or DNA), ammonium, phosphate, uric acid, volatile organic compounds, or other markers. The sensors can be any number of sensors capable of detecting data associated with the fluid. The sensors can include, but are not limited to biological, electrical, chemical, optical, temperature, ultrasonic, resistive, and/or MEMs sensors. The data from the sensors may be pre-processed to prepare the data for further analysis. For example, the data can be pre-processed to improve signal, reduce noise (for example, mechanical or electrical), and/or normalize data.

At a block 5612, the process 5600 can include analyzing the data. The data can be analyzed to determine any number of parameters, trends, or other characteristics associated with sensor data. One or more hardware processors can determine the parameters, trends, or other characteristics at block 5612. For example, a fluid can be an effluent in an ostomy bag. Sensors can measure biochemical markers associated with the effluent, including but not limited to sodium, glucose, and potassium or inflammatory markers, or any other markers disclosed herein. Certain changes in potassium and sodium in effluent can indicate hydration and/or dietary issues and/or certain disease states. The process 5600 can include analyzing the potassium and sodium levels to determine a trend of increasing or decreasing potassium and sodium levels. The analysis can be performed based on the sensor data related to the parameter of interest over a period of time collected by the monitoring devices, such as days, weeks, months, years, or longer.

At block 5614, the process 5600 can include analyzing the output of block 5612 to determine if a criteria associated with the data is met. For example, the output of block 5612 may be a parameter, trend, or other characteristic associated with sensor data associated with a fluid. One or more hardware processors can determine if the parameter, trend, or other characteristic meets a threshold criteria. The threshold criteria can include, but is not limited to a rate of change of a parameter over a period of time (for example, an hour, a day, a month, a year), a threshold value of a parameter, a threshold range, a number of times that a parameter fell outside of a threshold range, or any other suitable criteria. The period of time may be long enough to analyze long term trends. If the parameter, trend, or other characteristics pass the threshold criteria, then the process 5600 could move on to block 5616. If the parameter, trend, or other characteristics do meet the threshold criteria, then the process 5600 could repeat, going back to receiving data at block 5610. Optionally, the data could be displayed at block 5618 on a display, such as a mobile device of the user. In this manner, a fluid can be continually monitored.

For example, a fluid can be an effluent in an ostomy bag. The output of block 5612 can include a trend of potassium values and/or a trend of sodium values over the course of a period of time, such as a month. A threshold criteria can include a number of times that sodium and potassium values fell outside of a range associated with proper hydration of a user of the ostomy bag over the course of a month. For example, the threshold could be 10 times that a user's level of sodium or potassium fell outside the range associated with proper hydration. Additionally or alternatively, the threshold criteria could be an increasing number of times over smaller periods that a parameter fell outside a threshold range. For example, the threshold criteria could be that sodium and potassium values fell outside of a range associated with proper hydration 50 percent more frequently in the last month as compared to previous months.

At block 5616, the process 5600 can include transmitting a signal based on the outcome of the determination at block 5614. The signal can include any number of alerts, data points, parameters, characteristics, trends, or recommendations associated with sensor data. The signal can be transmitted to a display, for example, at block 5618. Additionally or alternatively, the signal can be transmitted to any number of external devices. For example, if a threshold criteria is met at block 5614 that indicates increasing instances of dehydration, a signal can be transmitted to a clinician device, care giver device, or user device. Advantageously, the process 5600 (and in particular the use of the threshold criteria and continual monitoring) can allow a care-giver to better treat a user of a monitoring device 5114 or device 5200 because it can alert care-givers to long term trends in a user's health where the monitoring device 5114 or device 5200 are placed to measure a user's fluids.

### Examples of Ostomy Bag

For example, an ostomy wafer or ostomy bag can include one or more monitoring devices for measuring biochemical parameters associated with the contents of the ostomy bag.

An ostomy bag can be a medical bag that collects human waste (either stools, urine, or both) from patients who cannot excrete waste naturally due to medical issues, which include, among others, cancer, trauma, inflammatory bowel disease (IBD), bowel obstruction, infection and fecal incontinence. In such cases, a surgical procedure is performed whereby a waste passage is created. This waste passage can be the ureter (called an urostomy), the small bowel or ileum (called an ileostomy, part of the small intestine) or the large bowl or colon (called a colostomy, part of the large intestine), which may be diverted to an artificial opening in the abdominal wall, thus resulting in part of the specific internal anatomy, to lie partially outside the body wall. This procedure can be referred to as an ostomy, and the part of the waste passage which is seen on the outside of the body can be referred to as a stoma.

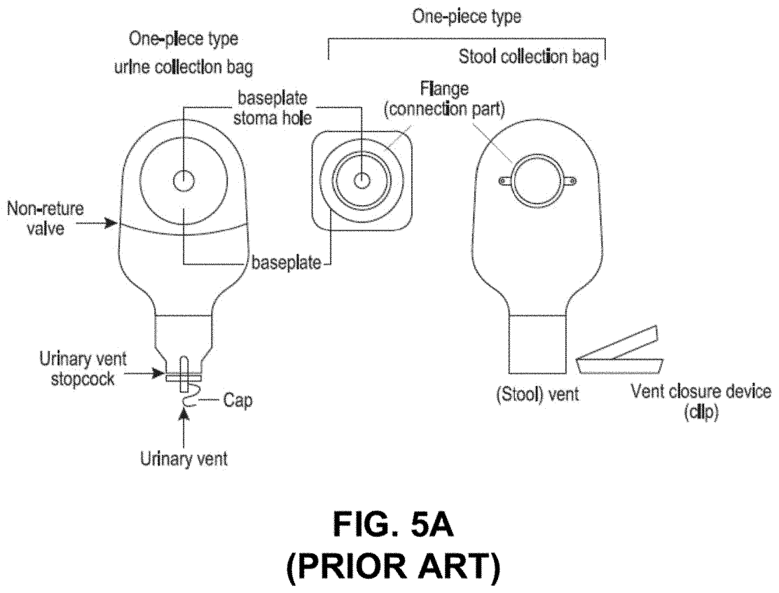

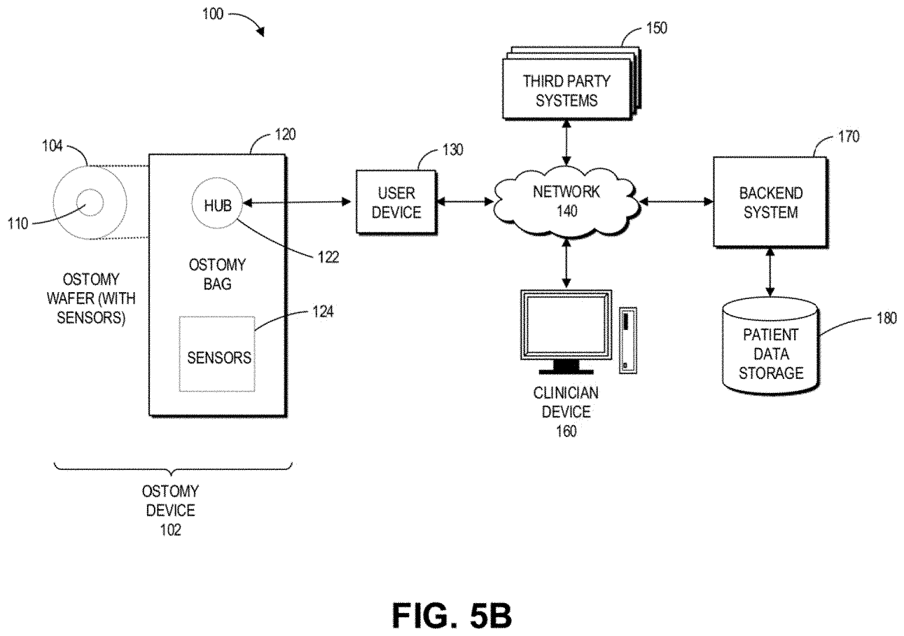

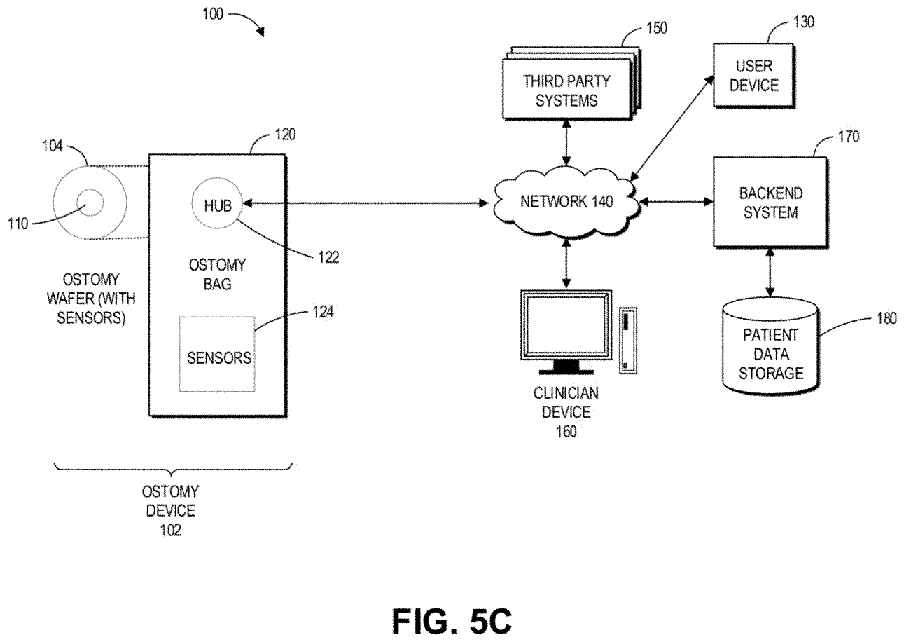

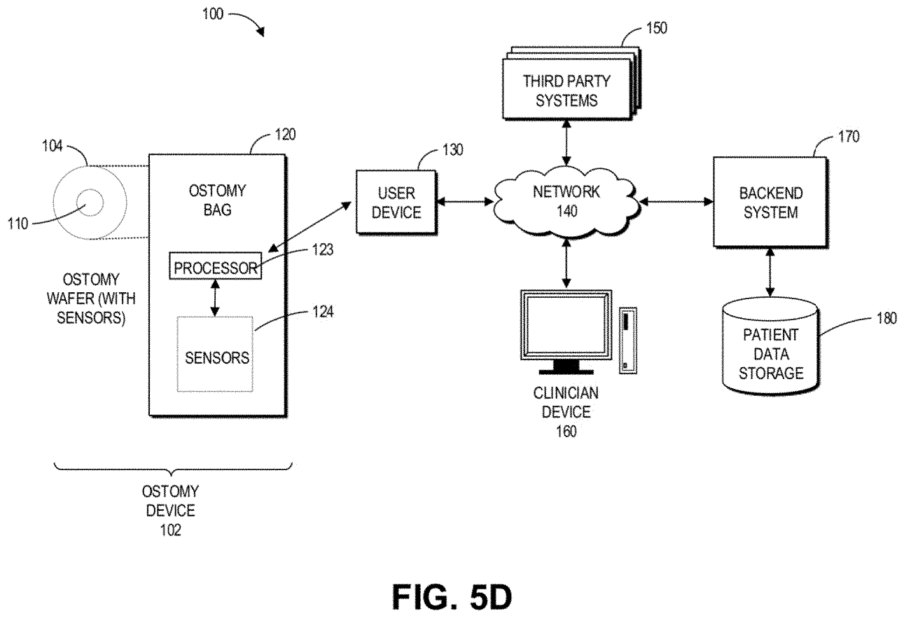

A prior art image of example ostomy bags is presented in FIG. 5A. In FIG. 5A, two ostomy bags are shown. These bags include a one-piece bag to the left and a two-piece bag to the right. The one-piece bag (on the left) has a baseplate (also sometimes referred to as a faceplate or called an ostomy wafer or simply wafer) already attached and integrated onto the bag. The two-piece bag has a separate wafer and bag (and thus includes an attachment or flange). In the case of the one-piece bag, it is usable only once, and when it is time to change the bag, the full appliance needs to be disposed. In the case of the two-piece bag, the bag can be disposed without having to take off the wafer. Some people prefer this two-piece set-up, leaving the wafer on their bodies while removing only the bag, as removal of the wafer (which may contain a high-tac adhesive) can be a form of mechanical strain on the skin, which some prefer to avoid. When the bag is worn on the user, the wafer side in the one-piece bag, or the wafer-interfacing side of the two-piece bag, can face the user's body. The wafer can sit around the stoma (thus, the stoma sits in a stoma hole in the wafer) and can be made from a biocompatible hydrocolloid or hydrocolloid adhesive-based material, which are both skin friendly and so can stick to the skin easily once the stoma is in place through the stoma hole. Many other example wafer and bag materials are described in greater detail below. Both diagrams are examples of drainable bags, in that they have vents at the bottom of the bag for the patient to remove the waste when it is time to empty their bags. Some bags do not have a vent and so cannot be drained. Thus, when full, such bags are disposed without the function to be able to drain them. The average wear time of an ostomy bag/pouch can be 1-3 days or 3-5 days. The average wear time of a baseplate can be about 3-5 days.

The type of waste released by patients with the three different forms of ostomies (urostomy, ileostomy, and colostomy) can be different. Urostomy waste includes urine, ileostomy waste can include stools of porridge-like consistency, and waste from colostomy patients can include firm stools. The size of the stoma that is created by the stoma surgeon may be determined by the specific type of ostomy that the patient has. For example, a colostomy is the divergence of the colon (large intestine) to the opening in the abdominal wall and hence the stoma size (for example, the diameter) may be expected to be quite large. This is in contrast to an ileostomy patient, who would have his/her ileum (part of the small intestine) diverted to an opening in the abdominal wall. Because of the smaller size of the small intestine, the stoma size is likely to be smaller.

Currently bags in the medical bag industry (which includes ostomy bags, blood bags, saline bags, catheters, etc.) function solely as plastic bag type collection vessels which can be emptied and re-used, or disposed and replaced by a new one. Other than that, they have no advanced functionality or uses, for example clinical diagnostic capabilities. Thus, for example, analytical urine and stool tests are currently conducted in a lab facility by the physical collection of samples from the patient, which are subsequently sent to various diagnostic labs for clinical laboratory analysis.

This disclosure describes several different example bags and wafers that can include sensors and optionally electronics. The electronics on the bags and/or wafers can perform a significant amount of analytical analysis (for example, calculation of at least some of the leak and/or skin irritation detection metrics disclosed herein). The sensors and electronics on the bag and/or wafer can transmit sensor signals (which can be unprocessed and/or minimally processed or conditioned signals) to a back-end system (such as cloud servers) for calculation of the metrics (for example, the temperature and/or capacitance change). With systems incorporating such bags and wafers, the measurement of other metrics can be done within the bag itself (optionally together with an external device such as a patient's phone), without the need for third party intervention, such as a lab, to conduct the analysis. Thus, this disclosure describes some examples of a “lab on a bag.” The bag can effectively be able to give each patient as well as his/her physician and/or nurse and/or caretaker, in-situ patient clinical information.

An example of such clinical information can be electrolyte levels such as sodium (Na⁺), calcium (Ca²⁺), or potassium (K⁺) levels, the loss of which can be indicative of patient hydration levels as well as acting as markers for diabetes, renal and liver dysfunction as well as cardiac and other diseases. Another clinical marker that may be used on bags herein is the pH level, for example in urine, which can give indication of UTls (Urinary Tract Infections) as well as ketosis and severe diarrhea. Other types of substances in the output can be monitored, such as presence of drugs.

Other metrics can be of incredible value to both the patient and his/her medical team in charge, as well as possible care giver. In response to this, the bag and/or wafer can also measure the physical information associated with events which occur on a daily basis in the lives of ostomy patients. This physical information can encompass data on the fullness of the bag as well as monitoring the volume of output in the ostomy bag, the flow rate in the effluent/output, its physical phase and the viscosity of the effluent, and finally peristomal skin irritation and leakage of the effluent, both around the site of the stoma and in the hydrocolloid wafer. A brief overview of examples of these metrics follows.

Bag Fill and Volumetric Measure:

Data and indicators regarding the fullness of the bag can be useful metrics for patients, providing early indication that his/her bag needs to be emptied, which can prevent the patient from potentially unfortunate and embarrassing incidents such as overfilling of the bag and can prevent the effluent from contacting the skin around the stoma site thus causing irritation or infection. Such incidences can impact patients socially and psychologically. Further, volumetric output can have a strong correlation to the patient in terms of their diet and hydration and therefore can be a good indirect indicator of the functionality of the GI (Gastro Intestinal) system and its ability to absorb nutritious components such as vitamins, proteins, glucose, minerals, and the like whilst being indicative of its throughput in removing the waste from the patient's body. Thus, a quantitative measure of the volumetric output from the stoma can indirectly give clinical guidance of the functioning of the GI system.

However, the output of each patient can be a very subjective metric, with some patients having significantly more output and others significantly less. Linearity may not always be the case in the relationship between input and output, with some patients having significant output in comparison to what is going into their bodies. Thus, the combined information of the input of the patients with their output, could lead to early signs of for example dehydration (for example, by losing significantly more water through the measured output than that which is going into the body via fluid intake).

A mobile application and/or web site can be provided to patients, which can include a platform of different trackers such as food and hydration trackers. With the application optionally being able to record metrics such as diet and hydration (via user interaction and trackers within the app) and the bag sensor(s) able to indicate the volume in the bag, this integrated platform can work together to give early signs of dehydration, dietary issues or even GI dysfunction in patients. Dehydration can be a significant metric because it is one of the most common reasons why patients are readmitted into the hospital in the first three months following ostomy surgery. Thus, providing features that can help patients become aware of their output can enable patients to better monitor and prevent dehydration, significantly improving quality of care and life while at the same time potentially reducing the post-operative costs associated in hospital re-admissions following initial stoma surgery.

Flow rate, the physical phase and the viscosity of the effluent:

Knowledge of the physical phase (including solid, semi-solid, liquid, and gas) of the effluent that is coming out of the bag can be clinically significant. In the case of urostomates and colostomates, the phase of the output can be generally fixed for both groups of patients, with the output being of liquid and solid phases, respectively. However, in the case of ileostomy patients, the output may be of porridge-like consistency, meaning it can be a mix of solid, liquid, or semi-solid. Moreover, both colostomy and ileostomy patients may have gas in the output. The knowledge of the phase of the output can give early signs of dehydration, functionality of the GI tract of the patient, and information about the lifestyle of the patients such as their dietary habits or hydration habits. Combined with the mobile application discussed above, clinically significant data and events can be determined and relayed to doctors rapidly. Moreover, detection of gas output can enable a more accurate calculation of bag fill, as discussed below in more detail.

Skin Irritation and leakage of the effluent around the stoma:

Leakage as a phenomenon, is particularly common with patients who have more fluid-like output, but can also occur with colostomy patients who have more firm output, through so-called “pancaking” of the stool around the stoma. Leakage can occur when the effluent/output of the patient does not entirely enter the bag. Instead, some of it bypasses the bag and starts to accumulate between the adhesive side of the wafer (skin-side facing) and the skin surrounding the stoma (also called the peristomal skin, which lies behind the wafer). The output encompasses biological and chemical enzymes, which when in contact with the skin for long periods of time, and as a function of their accumulation, can start to “erode” and thus irritate and scar the skin. The method by which skin is irritated in this scenario can be called Irritant Contact Dermatitis (ICD) or Incontinence Associated Dermatitis (IAD). For ease of description, this specification often refers to ICD and IAD interchangeably.

Leakage can be caused by a number of reasons, with some of the main reasons being the loss of tackiness of the hydrocolloid adhesive as a function of long wear times or sweat and/or moisture accumulation between the wafer and the skin behind it. The accumulation of this enzymatic output, behind the wafer, can also promote erosion of and can destroy the hydrocolloid. In doing so, this erosion can break down the adhesive too, destroying its tackiness and therefore ultimately making it redundant. Long wear times are very common with ostomy bags, with 3-5 days being the average wear time per patient before disposal to utilize a new bag. Thus, it can be imagined that over this long period of continuous wear, the hydrocolloid is likely to be exposed to significant amount of moisture, resulting in its ultimate inability to be utilized without leaking.

Moisture and sweat can also act as catalysts to exacerbate the symptoms of leakage because as these forms of moisture start to saturate the hydrocolloid, which has a maximum saturation limit, beyond which it cannot absorb further moisture, then they effectively prevent the hydrocolloid from absorbing the leaking effluent. As a result, the leaking effluent accumulates in between the peristomal skin and the back of the wafer, causing ICD.

ICD is a major concern and issue with a large number of patients, but so far the interventions made by the major bag companies to prevent leakage and subsequently skin irritation, include the utilization of products such Eakin seals which limit the leakage or wipes that form a protective barrier that protect the skin from damage of the adhesive, effluent and enzymes or integration of components like ceramide into the barrier to maintain good skin health and maintain good peristomal skin health. Despite these interventions, many patients are still struggling with peristomal skin complications. One disadvantage that patients face is the lack of sensation of the leakage occurrence. By the time the patient realizes that leakage has occurred, it can become too late because the active enzymes species may have already done significant damage to their peristomal skin. The skin irritation that occurs can be on multiple levels, which WOCNs (Wound Ostomy Care Nurses) can assess via the DET (Discoloration, Erosion and Tissue Overgrowth) score. This scoring system is described as an ostomy skin tool utilized by nurses as a standardized way of assessing the peristomal skin conditions and complications in ostomy patients. This scoring tool is scored for skin irritation promoted by chemical irritation which encapsulates IAD or ICD, mechanical trauma (due to frequent change of the bag wafer), disease related irritation and infection related irritation, as seen in the previous citation. The infection around the stoma can be a symptom of the initial skin irritation coupled with moisture and the presence of sweat.

As yet, based on inventor knowledge, there has been no commercial interventions to provide a technological solution which can indicate the in-situ occurrence of leakage or the saturation and/or breakdown of the hydrocolloid or potential skin irritation at an early stage. However, example devices and algorithms described herein can give users a warning to change their flange/wafer and thus take preventative action to minimize their skin conditions worsening.

Further, there has been no commercial technological solution, based on inventor knowledge, for the detection of the volume in the bag, assimilation of the physical phase in the bag and the flow rate, where temperature is being used as a marker. Solutions to be able to detect these metrics from a technological perspective, with an overall motive to communicate this information (for example, in real-time) to a variety of different stakeholders (for example, patients, nurses, doctors, care givers, care takers) via a smart phone or smart tablet platform as illustrated further below, would be of great value to the healthcare and patient communities.

An example smart ostomy bag (or “smart bag”), which can also encompass a wafer, can have integrated sensors that can track one or more in-situ physical events inside the bag. These events can include volumetric analysis, flow rate, physical phase of the effluent, viscosity of the effluent, possible skin irritation, and/or leakage occurrence around the stoma and saturation of the hydrocolloid. The smart bag can also track more detailed clinical/analytical metrics of the bag such as electrolytic measurements, pH, and other markers, which be explained in further detail below.

One physical marker that can allow for the detection of some or all of the metrics described above is heat/temperature. The following section will explain why heat can be a relevant marker in order to detect one or more metrics of interest.

As mentioned previously, peristomal skin irritation is one of the top-ranked complications for ostomy patients, which can be caused by frequent change of the wafer, allergy, folliculitis, or leakage of the skin barrier/wafer (a leakage can occur when the stoma output seeps between the skin and the skin barrier/wafer, which may eventually extend outside of the skin barrier/wafer).

Despite the variety of factors that cause ICD, which can collectively be termed irritants, each of these factors can lead to an increased subcutaneous blood flow, and resultantly, an increased skin surface temperature. Though specific clinical data on peristomal skin temperature is not available in literature, other studies on chronic wounds and ulcers have shown evidence of a 3-4° C. difference in skin temperature between the irritated skin and the contralateral unaffected reference skin irritation. Therefore, the in-situ monitoring of the peristomal region skin surface temperature, as well as a region further away from this periphery (in order to have an un-irritated reference area of measure), can provide information about the skin health and can indicate early signs of skin irritation.

Since stoma output can be associated, at least initially as the output leaves the stoma, with internal body temperatures (at or about 37° C.) which is higher than the external skin temperature (specifically the abdominal skin surface) (about 32-35° C.), temperature can also be utilized as a marker to warn of leakage occurrence behind the skin barrier/wafer and therefore to alert the on-coming of early-stage peristomal skin irritation. When the leakage occurs, it would be expected that the temperature in the wafer may increase very rapidly—even appearing to be an instantaneous increase. This rapid or instantaneous temperature change can be monitored as a function of the leakage occurrence to detect the leakage in-situ.

The wafer of the ostomy bag made with hydrocolloid-based materials can have advantages including but not limited to: 1) it adheres to the skin surrounding the stoma, whether it is moist or a dry skin site, 2) in the case of wound exudates, which are a very common occurrence in ostomy applications, the hydrocolloid dressing absorbs fluids and swells, protecting the wound, causing less pain and faster healing and 3) given that in ostomy applications most bags are commonly changed after about a 1-1½ day, 1-3 day, or 3-5 day period in the USA (commonly about 1-2 days in the UK), and the baseplates being changes after about every 5-6 days, the wear life of the hydrocolloid dressing can be sufficiently long such that, once worn, the dressing needs not be replaced in between bag changes, causing less disruption to the wound.

Given that the hydrocolloid absorbs exudates as well as moisture from the body, for example sweat, it is expected that it will expand as a function of the absorption of the fluids. The expansion of the hydrocolloid as a function of the absorption is suggestive of a change in temperature between the hydrocolloid adhesive and the peristomal region as the hydrocolloid effectively moves away from the skin as a function of the exudate absorption. Therefore, the route to detect the saturation of the hydrocolloid, can be via detecting the temperature change as a function of time, which can give early indication of the saturation of the hydrocolloid. This can be important as many patients do not have the sensation of leakage or of the hydrocolloid saturating until they can visually see or feel the flange detach off their bodies, which occurs naturally as a function of the reduced tackiness of the hydrocolloid adhesive.

Apart from temperature, another useful marker for detecting one or more metrics of interest, via the wafer or bag, can be the pH. The pH can be useful due to the leakage occurrence of the exudate and its contribution to the saturation of the hydrocolloid wafer. Given that the effluent contains enzymes of a biological and chemical nature, and the fact that they are able to erode the hydrocolloid and cause chemical damage to the skin, is suggestive an acidic or alkali nature of the effluent. Essentially the skin chemistry as well as the nature of the hydrocolloid wafer is changing as a function of the chemical and/or biological attack. By detecting the change in pH of the hydrocolloid as a function of the leakage occurrence, or its saturation and/or alternatively detecting the pH of the skin as a function of the enzymatic attack, a powerful combination of sensors (temperature and pH) can give early indication of leakage/skin irritation/saturation of the hydrocolloid wafer. By embedding (for example) a thread-based microfluidic pH sensor into the wafer, oversaturation and leakage can be detected. Of course, pH monitoring is optional.

Heat/temperature as an example marker for measuring metrics from the front (and potentially the back) of the main body of the bag will be described in greater detail below.

Some ostomy bag can include a volumetric sensor, based on a resistive flex sensor, which can measure the volumetric fill in bags and warn the patients for the draining points (for example, the times to empty their pouches). The nature of the flex sensor causes it to suffer from noise because of patients' natural movements (sitting, standing sleeping, running) and movements of the content within the ostomy bag.

As mentioned above, effluent is likely to be initially at internal human body temperature (at or about 37° C.) which is higher than the external skin (specifically the abdominal skin surface) temperature (about 32-35° C.). Therefore, the utilization of heat/temperature as a marker to understand the volumetric fill in the bag can be used to determine the volume in the bag. The effluent is likely to be the warmest when it exits the stoma, and as it travels from the top of the bag to the bottom of the bag where it settles, it may gradually cool down. The movement of the effluent from the top of the bag to the bottom of the bag, as well optionally as the possible settlement of effluent, can be heat mapped and thus be indicative of the volume in the bag. 2D or 3D heat mapping of the bag can be used to understand the volumetric activity in the bag.

Temperature measurements can permit visualizing the thermal signatures and heat patterns across the front and/or back of the bag, as the effluent enters the bag. The thermal signatures of the effluent can therefore be traced from the point where the effluent enters the bag to the point where it settles. Given that the output can be of different physical forms depending on the type of ostomy a patient has, such as urostomy (fluid-urine), colostomy (firm stool-solid) and ileostomy (porridge like output semi-solid/solid-liquid), the flow rate can be visually mapped by understanding the rate at which an array of thermal sensors is fired up, as the effluent crosses their path whilst heat is evolving/dissipating from the waste at the same time.

Heat dissipation, or more specifically rate of the heat dissipation, and cooling, can vary between the different physical phases, as can the flow rate. The rate of heat dissipated can depend on the heat capacities of the different phases as well as if the waste is in motion or stagnant. The flow of each phase can depend on the viscosity, with the liquid urine samples likely to be less viscous as the particles in liquid are to some extent free-flowing, allowing this phase to flow and travel quickly into the bag, and cross the path of the thermal sensors very fast. In the case of solid waste, the flow rate can be significantly slower due to the less free-flowing particles in the phase, and hence where an array of temperature sensors would be present, this phase is likely to cross the path of the thermal sensors more slowly. Therefore, it is possible to tell from the rate at which essentially an array of thermal sensors fires up—for example, the sensors' response time to the rate of movement of the effluent whilst it is entering the bag at internal body temperature and crossing the path of the array of thermal sensors—the viscosity and therefore the phase of the effluent. The timeframe of how long the thermal signature of the volumetric output lasts can also allow for indirectly determining the viscosity and phase of the effluent (such as liquid, solid, semi-solid, and gas). It would be expected (depending on the rate of heat dissipation) that the temperature of the output may drop back to baseline within a certain timeframe, but this timeframe can be different for different phases and viscosities.

The integration of arrays of thermal sensors into ostomy bags and/or wafers can aid patients as well as their care givers, nurses and specialist doctors to manage peristomal skin complications and to take early action to prevent the skin condition of the ostomy patient from worsening. Further, patients and caregivers may be able to understand more about the patient's output and the function of their GI system. Specific temperature sensor technologies, as well as other sensor technologies, for wafers and bags are described in greater detail below with respect to the drawings.

The smart ostomy bag can also detect the volume/fill inside the bag, such as by using the same thermistor technology mentioned above. The thermistor technology described above can detect the volume from the thermal signature of the effluent output; such as by placing the thermistor sheet in front of or in back of the bag (for example, in either a front wall or a back wall of the bag). The time frame of the thermal signature of the volumetric output can indirectly indicate viscosity and eventually phase of the effluent (such as liquid, solid, semi-solid, and potentially even gas).

The thermistor based sensor technology can have a two-fold functionality in the smart bag: 1) indicating skin irritation and leakage in the peristomal region and 2) indicating volume fill in the bag as well as phase of the effluent released. Both data sets can be generated based on heat. Below is an explanation of the processes and principles used by the device to generate output for each of these measures.

Because the transduction principle of the thermistor sheet can be based on temperature change and not on bending as the flex sensor is in U.S. Pat. No. 9,642,737, the thermistor technology can be more immune from noise caused by movement and therefore a new candidate for volumetric indication in the bag. Additionally, in example implementations where this thermistor sheet is placed at the front of the bag, the sheet can detect the temperature distribution/diffusion of the content within the bag and also the flow pattern of the stoma output. This can further allow analyzing the rheology properties of the stoma output, and potentially allows identifying the phase of the output.

The temperature readings themselves can be derived from resistance readings of the thermistors at a particular temperature as a function of time. The thermistor can be a semiconductor based device that changes its electrical resistance as a function of applied temperature. The resistance value can then be converted to a temperature value via the Steinhart-Hart equation:

$$\frac{1}{T} = A + B ln ( R ) + {C [ ln ( R ) ]}^{3}$$

where T is the temperature (in Kelvin), R is the resistance at T (in ohms), and A, B, and C are the Steinhart-Hart coefficients which can vary depending on the type and model of thermistor and the temperature range of interest.

The sensors can send data to an electronic hub, which can packetize the data and send the packets to a cloud server and/or to a mobile application on a user device. The mobile application can read the wireless packets and convert them to their appropriate data types. The mobile application can also be in electrical communication with the cloud server to download the data. The mobile application can output, for presentation to a user, a map of the heat distribution throughout the wafer and the front side of the bag, a temperature versus time scattered plot, and/or as visual representation of the total volume of output in the bag.

### Example Ostomy Monitoring System

In FIGS. 5B-5D, a schematic overview of an ostomy monitoring environment 100 is provided in which an ostomy device 102—as well as optionally a patient (not shown) using that device 102—may be monitored. In this environment 100, a hub 122 of the ostomy device 102 is shown in communication with a user device 130 (see FIG. 5B), which can transmit data from the hub to a backend system 170 (such as a remote server or cloud server) over a network 140, or directly with the backend system 170 over the network 140 (see FIG. 5C). The user device 130, the backend system 170, and other devices can be in communication over the network 140. In some cases, such as shown in FIGS. 5B and 5C, the user device 130 can download processed data from the backend system 170 after the hub 122 transmits the data to the backend system 170 for further processing (although in FIG. 5C, the backend system 170 can communicate directly with the hub 122 instead of through the user device 130). These other devices can include, in the example shown, a clinician device(s) 160, and third party systems 150. The ostomy monitoring environment 100 depicts an example environment, and more or fewer devices may communicate with the ostomy device 102 in other systems or devices. The ostomy monitoring environment 100 can enable a user and others (such as clinicians) to monitor various aspects related to the user's ostomy device 102, such as ostomy bag fill, leaks, and skin irritation. The ostomy monitoring environment 100 in FIG. 5D can differ from the ostomy monitoring environment 100 in FIG. 5B by being hub-less, that is, a processor 123 in communication with the sensors 124 are also in communication with the user device 130. The user device 130 can download processed data from the backend system 170 after the bag processor 123 transmits the data to the backend system 170 for further processing.

The ostomy device 102 can be a one-piece or two-piece device including an ostomy wafer 104 and an ostomy bag 120.

The ostomy wafer 104 can include a patient-facing side that has an adhesive pad, flange, or the like that attaches to a patient's skin around a stoma 110 and a bag-facing side that is opposite the patient-facing side. The stoma 110 can include any stoma disclosed herein, for example, an aperture or hole in a patient's abdomen (or other location) resulting from a colostomy, ileostomy, urostomy, or other similar medical procedure. The ostomy bag 120 can removably attach to the bag-facing side of the ostomy wafer 104 (such as via adhesives or a Tupperware click mechanism) and receive and store output (for example, effluent) from the stoma 110. The ostomy bag 120 can be flexible so that when the bag 120 can be substantially flat when empty and can expand as effluent enters the bag 120. Once the ostomy bag 120 has reached its designed capacity, the patient (or caregiver) may remove the ostomy bag 120 from the ostomy wafer 104, discard and/or empty it, and attach a new ostomy bag 120 (or clean and reattach the old ostomy bag 120). In another example, the ostomy bag 120 is provided or sold together with the ostomy wafer 104 as a single device, with the ostomy wafer 104 integrally formed with the ostomy bag 120. The ostomy bag 120 collects human waste (such as stools and/or urine) from patients who cannot excrete waste naturally due to medical issues, which span from cancer, trauma, inflammatory bowel disease, bowel obstruction, infection, and incontinence. In such cases, a procedure is performed where a waste passage is created (colostomy, ileostomy, or urostomy) and diverted to a section of the abdominal wall. The ostomy bag 120 can be made of non-porous sterile plastic materials such as, but not limited to, polyvinyl chloride, polyethylene, ethylene vinyl acetate, polypropylene, and copolyester ether.

The ostomy bag 120 can include one or more sensors 124 and optionally a hub 120, which can be located on a side facing away from the wafer 104. The sensors 124 can include any of the sensors described herein. For instance, the sensors 124 can include a plurality of temperature sensors, capacitive sensors, a camera (infrared or visible light), a gas sensor, a magnetic sensor such as an AMR sensor, and/or microfluidic sensor(s), among others. The bag 120 can include multiple layers. One or more sensor layers may be provided in which sensors are embedded or otherwise attached. Different types of sensors may be on different layers, or different types of sensors may be on a single layer. The sensors can also be located on the same and/or different sides of a single layer.

The ostomy bag 120 can include a measurement sheet. The side of the ostomy bag 120 facing away from the wafer 104 can include the measurement sheet. The measurement sheet can include a plurality of layers (such as layers made of polyimide, polyurethane, or the like). As will be described in greater detail below, four or two layers can be used. Other numbers of layers can be used. A layer of temperature sensors and/or a layer of capacitive sensors, for instance, may be provided that detects temperature and/or capacitance changes as effluent enters the bag 120 and disperses about an interior of the bag 120. The temperature and/or capacitive sensors may each be arranged in a matrix or matrix-like arrangement. A processor, whether in the hub 122 (discussed below), the user device 130, or the backend system 170, can process the temperature and/or capacitance data obtained from the temperature and/or capacitive sensors to detect leakage and/or skin irritation metrics, such as an increase in temperature and/or bag fill. Electronics in communication with the sensors can also be provided on one or more of the layers. Other examples of the sensors with respect to the bag are discussed in greater detail below.

The ostomy wafer 104 can be a flexible sheet with one or more layers, and optionally, multiple layers including one or more sensor layers. The layers can be made of the same or similar materials as the layers of the bag 120 described above. One or more of the layers of the ostomy wafer 104 may include one or more of the following sensors: temperature sensors (such as thermistors, temperature sense integrated circuits (ICs), thermocouples, infrared (IR) temperature sensors, etc.), capacitive sensors, flex sensors, odor sensors, microfluidic sensors, leak sensors, combinations of the same, or the like. The ostomy wafer 104 can also be a moldable barrier.

The sensors (such as temperature sensors and/or other types of sensors disclosed herein) of the ostomy wafer 104 can be disposed in a sensor layer (described in detail below). The sensor layer can have a similar or the same shape outline as the ostomy wafer 104. For example, if the ostomy wafer 104 is shaped like a donut or annulus, the sensor layer may include a generally annular shape. The sensor layer can also have a shape that differs from the general shape of the wafer 10, such as a partially annular or partial ring shape. Optionally, the ostomy bag 122 can include a carbon filter port to allow gas to escape. An optional gas sensor placed on or near the port can detect a characteristic about the gas, such as the pungency of the gas to determine the status of the user's gut.

The ostomy wafer 104 can be any size. The size of the ostomy wafer 104 can depend on the type of stoma that the wafer 104 is used with. For example, a colostomy stoma can be larger than a urostomy stoma. Thus, the ostomy wafer 104 can be sized larger for some colostomy stomas than for some urostomy stomas. The ostomy wafer 104 may be a “one-size fits all” wafer that has punch-out sections in the center for adapting to various different stoma sizes. The ostomy wafer 104 can also come in different versions, which have stoma holes 110 of different sizes to accommodate different stoma sizes.

The ostomy wafer 104 can also be in any of a variety of different shapes. For example, the ostomy wafer 104 can have a generally annular, ovular, or circular shape, such as a ring, donut, or the like. The ostomy wafer 104 can also have a more rectangular, oblong, or square shape (optionally with rounded corners).

As described above, the ostomy wafer 104 can be layered in structure to encapsulate the sensors. Encapsulation can improve fixation of the temperature sensors in position in the flexible sheet and/or reduce corrosion of the sensors by the external environment. As an alternative to encapsulation, the temperature sensors may be protected from corrosion by a coating, such as a conformal coating. Some example wafers (and bags, discussed below) can have at least one temperature sensor in a second region of the flexible sheet that is protected by a conformal coating.

As described above, the patient-facing side of the ostomy wafer 104 can have an adhesive side that adheres to skin around a stoma 110 and/or directly to the stoma 110. The adhesive can be a double-sided adhesive. The adhesive may be a hydrocolloid adhesive.

The sensors of the ostomy wafer 104 and/or the bag 120 can detect information based on the output of the stoma 110. The sensors can sense the constituents of the effluent or output of the stoma 110. Temperature sensors can be used to determine whether there is likelihood of inflammation at the site of the stoma and/or a leak. Temperature sensors may also be used to detect the phasing of the constituents, which can be used to determine, for example, how much gas and/or solid is in the bag. A capacitive sensor in the wafer 104 (and/or in the bag 120) may serve as a fallback, provide redundancy to, and/or supplement a temperature sensor to determine if there is a leak. For example, the temperature sensors on the wafer 104 can detect a leak due to the effluent not entering the bag for various reasons as described above in addition to overfill of the bag 120 (such as when the bag 120 is relatively empty but the adhesives on the wafer become loose). As another example, the temperature and/or capacitive sensors on the bag 120 can detect bag fill and output an indication of an imminent overfill or leak, before an actual occurrence of a leak. In another example, capacitive sensors can be used instead of temperature sensors to detect leaks or skin irritation.

If microfluidic sensors are used on the wafer 104 and/or the bag 120, the sensors can be used to detect electrolyte or inflammation markers within the constituents. This data can be used to show the user what he or she could intake or do to obtain a healthier balance of electrolytes and other chemical compositions in the user's body. An odor sensor can be incorporated into the bag 120 and/or the wafer 104 to determine whether there is bacterial growth in the digestive tracts. An inertial measurement unit (“IMU”) sensor, a form of positional indicator, can also be integrated into the bag 120 and/or the wafer 104. An optical sensor, such as a camera, may also be integrated into the bag 120 and/or the wafer 104 where the sensor looks down over the stoma and/or into bag in order to detect a degrading stoma, blood in stool, or etc. An audio sensor, such as a microphone, can be included in the bag and/or the wafer to detect gas output and/or bowel movement sounds. pH sensors may also be integrated into the bag 120 and/or the wafer 104 to determine the acidity of the constituents of the bag.

The ostomy wafer 104 and the ostomy bag sensor(s) 124 can collect patient data related to the stomal output and can transmit the data wirelessly or with wires to the hub 122 or a processor in electronic communication with the sensors. The hub 122 can include electronics that can facilitate one or both of (1) processing sensor data and (2) transmitting sensor data. For instance, the hub 122 can include a hardware processor, memory, and a wireless transmitter. The hub 122 can also optionally have a display for outputting data related to the sensors (such as an indication of a leak, bag fill, or the like). The hub 122 can also optionally include a speaker that outputs an audible warning indicative of a leak, bag fill, or the like.

The optional wireless transmitter of the hub 122 or of a bag that does not include a hub can send data received from sensors (wafer or bag) to a user device 130. The data can then be sent to a network 140, third-party systems 150, a clinician device 160, a backend system 170, or to a patient data storage device 180 (each of which is discussed in greater detail below). In order to preserve battery life, the wireless transmitter may be switchable to an active mode and idle mode. The wireless transmitter of the hub 122 or of a bag that does not include a hub can also send data received from the sensors on the wafer 104 and/or the bag 120 to the backend system 170, such as shown in FIG. 5C. The wafer 104 and/or the bag 120 can send data periodically, for example, over Bluetooth. The data transmitted by the hub 122 or the processor 123 of a bag that does not include a hub can include unprocessed, or conditioned (such as filtered, demodulated, and so on) signal data. The backend system 170 can process the received signal data to calculate the metrics disclosed herein, such as temperature and/or capacitance values, bag fill volumes, and/or leakage detection. The user device 130 and/or other devices can download the calculated metrics from the backend system 170. Performing the calculation on the backend system 170 can reduce the need for processing power in the hub 122, which can in turn reduce battery consumption and/or frequency in changing or recharging a battery in the hub 122.

The optional wireless transmitter of the hub 122 or of a bag that does not include a hub may include a near-field communication (NFC) reader and/or writer, a Bluetooth transmitter, a radio transmitter, or a Wi-Fi (802.11x) transmitter. The NFC reader and/or writer can be coupled to NFC antennas on the hub for communicating with NFC antennas on the bag 120 and/or the wafer 104 to receive sensor data from the sensors on the bag 120 and/or the wafer 104. The NFC reader and/or writer can have sufficient power or current (for example, with an output current up to about 250 mA) to receive data transmitted by the NFC antennas on the wafer 104 (and/or the antennas on the bag) when the bag 120 is filled to its apparent capacity and/or when the wafer 104 is separated from the hub 122 by a certain (for example, maximum) distance. The NFC reader and/or writer can serve as the main wireless communication tool with the sensors on the bag 120 and/or the wafer 104, and Bluetooth communication can optionally serve as a backup tool. Different wireless communication protocols can also optionally be used for transmitting data among the hub, the ostomy bag, and/or the wafer. The Bluetooth transmitter may include a Bluetooth module and/or a Bluetooth low energy (BLE) module. A Bluetooth module may be, but is not limited to, a Bluetooth version 2.0+EDR (Enhanced Data Rates) module. A Bluetooth low energy module may be a Bluetooth module such as, but not limited to, a Bluetooth version 4.0 (Bluetooth smart), a Bluetooth version 4.1, a Bluetooth version 4.2 or a Bluetooth version 5. The Bluetooth sensor module may include a Bluetooth module using IPv6 Internet Protocol Support Profile (IPSP) or any other later versions.

The hub 122 can be in various positions on the device 102. The hub 122 can be placed in many areas on the ostomy bag 120. The hub 122 can be placed in the front, the back, next to a gas filter (not shown), or the like. The hub 122 can also be placed in a pocket on the ostomy bag 120 or the hub 122 could be a replaceable feature on the ostomy bag 120. The hub 122 can also come in different forms. When the hub is removed from an ostomy bag 120 it can use previous collected data and carry over that data to the next subsequent ostomy bag 120 that it is placed upon. Hub removability can save money for the user.

The hub 122 can include a plurality of electronics, including but not limited to the wireless transmitters and/or receivers, motion sensor (such as a three-axis accelerometer), temperature sensors (such as far infrared (FIR) temperature sensors, ambient temperature sensor, and/or the like), camera module, lighting for the camera (such as LED lighting), a microphone (such as a microelectromechanical (MEMS) microphone), battery charging circuitry, and/or other electronics. The ambient temperature sensor, which can be any type of temperature sensor, can be mounted on a side of the hub 122 facing away from the bag and the patient. Temperature measurements from the ambient temperature sensor can approximate a room or ambient temperature, and/or serve as reference for the temperature sensors on the bag 120 and/or the wafer 104. The microphone can record audio information related to the stomal output and/or monitor the metrics related to the stomal output (for example, gas output, bowel movement, or others).

The user device 130 can be any device with a processor and a wireless receiver that can communicate with the hub 122 or the processor 123 of a bag that does not include a hub. For example, the user device 130 can be a phone, smart phone, tablet, laptop, desktop, audio assistant or smart speaker (such as an Amazon Echo™, Google Home™ Apple HomePod™, or the like), television, or the like, that may pair automatically to the wireless transmitter and may include a mechanism that advises the user of the existence of a wireless link between the wireless receiver and the wireless transmitter. The user device 130 may have software and algorithms to process the data to show the user the status of the fill of the bag, the nearest restroom, nearest sources of electrolytes, nearest source of food, patterns and contents of discharge, hydration levels, and recommendations to improve the user's condition. The user device 130 may also transmit the data wirelessly to a network 140. The network 140 can be a local area network (LAN), a wide area network (WAN), the Internet, an Intranet, combinations of the same, or the like.

The third-party systems 150 can be a data processing tool/feature; backend servers for audio assistants; or fitness trackers, personal health monitors, or any third party systems that can use or manipulate the data collected by the device 102. These third-party systems 150 may also include algorithms and software to calculate and process the data.

Third party systems 150 and audio assistants can fetch data from the ostomy device 102 to announce reminders or alerts for the user such as to empty the bag, change the bag, change the hub, intake or stop intaking certain types of food, intake water, and/or providing periodic check-ins. Other third party systems may use data collected from other users to create a better feedback system or to identify patterns within a demographic of ostomy patients and/or bag users.

The clinician device 160 can be a data processing tool or monitoring program used by a clinician. These clinician devices 160 may receive data from the device 102 to provide a remote clinician to diagnosis the user, recommend actions to the user, or function as an augmented reality system for the clinician. These clinician devices 160 may also include algorithms and software to calculate and process the data.

The backend system 170 (such as cloud servers) can also use algorithms and software to perform data processing. For instance, the backend system 170 can process any data received from the sensors on the wafer and/or bag and return information based on that processing to the user device 130 or other devices. Another optional feature is an inclusion of a patient data storage system 180. From here the backing system can send the data to the patient data storage wirelessly or the patient data storage can access the data from the network 140.

Algorithms and software can show when the user should replace the bag, alert the user when the bag is nearly full or when there is a leak in the wafer or bag. Software features include, but are not limited to, identifying the nearest restrooms within the user's radius, the volume of the user's bag, alarms for different fill levels, a hydration and electrolyte tracker which calculates the user's recommended daily hydration goal with an algorithm. The hydration and electrolyte software can notify the user based on their effluent output or constituents what his or her dietary needs may be throughout the day.

### Example Ostomy Bags and Bag Layers

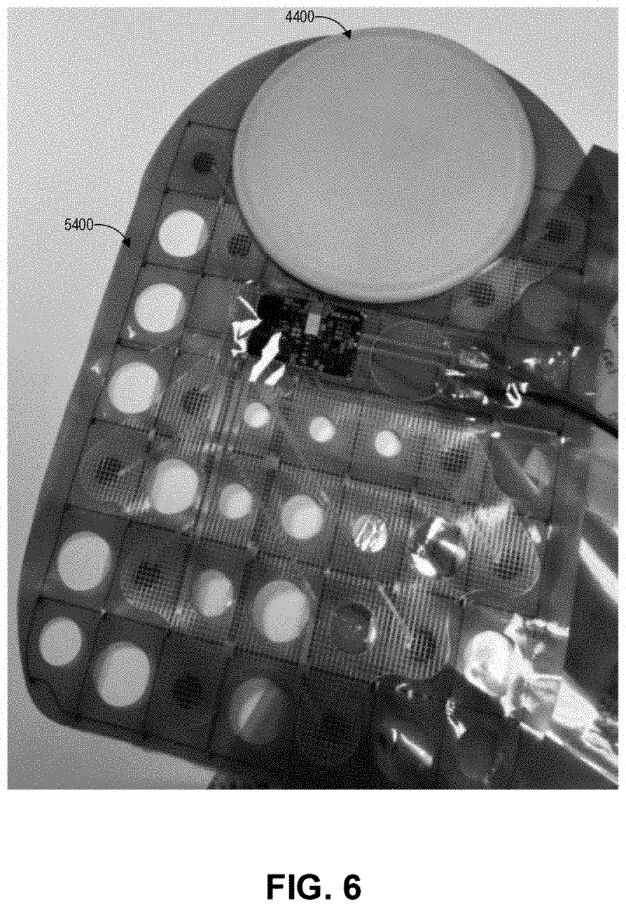

FIG. 1D illustrates an example ostomy bag 102, which may be used in conjunction with a monitoring device, such as the monitoring device discussed with reference to FIGS. 1A-4. The monitoring device disclosed herein can be placed inside (for example, with effluent contents of the ostomy bag), be part of, coupled to (for example, adhesively, or via hook and loop Velcro dots, attached to) the ostomy bag 102 or a hub 4400 (see FIG. 6), which may be coupled to the ostomy bag 102.

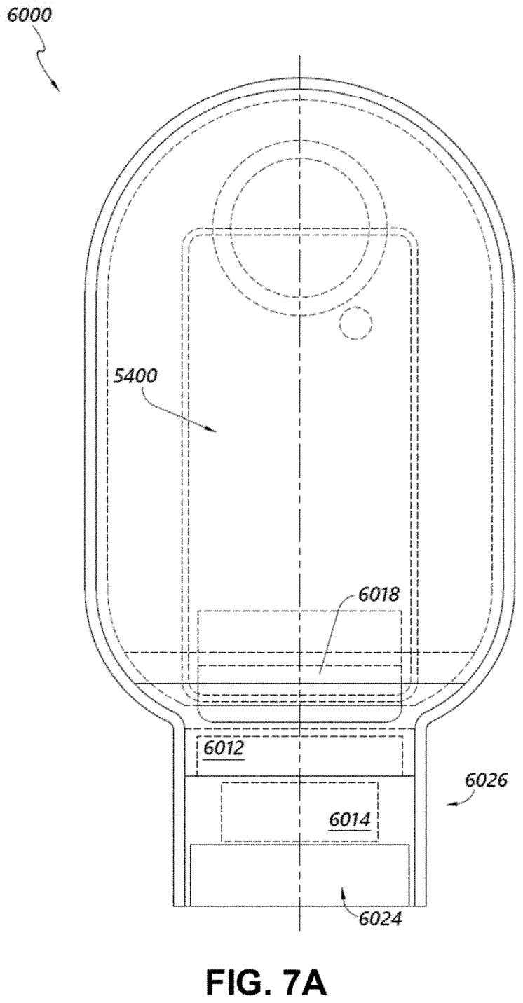

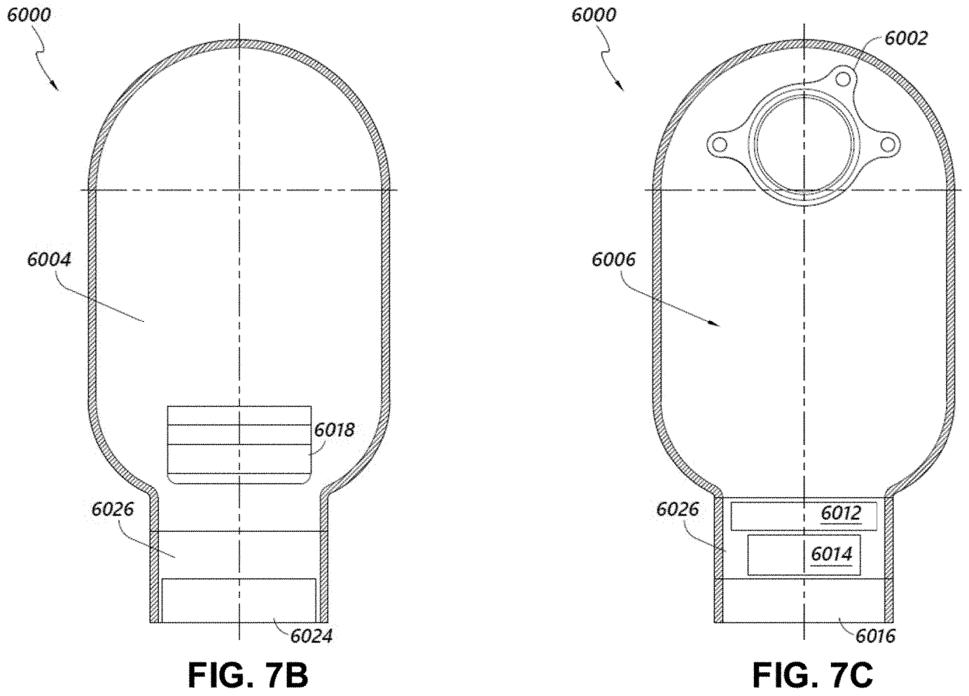

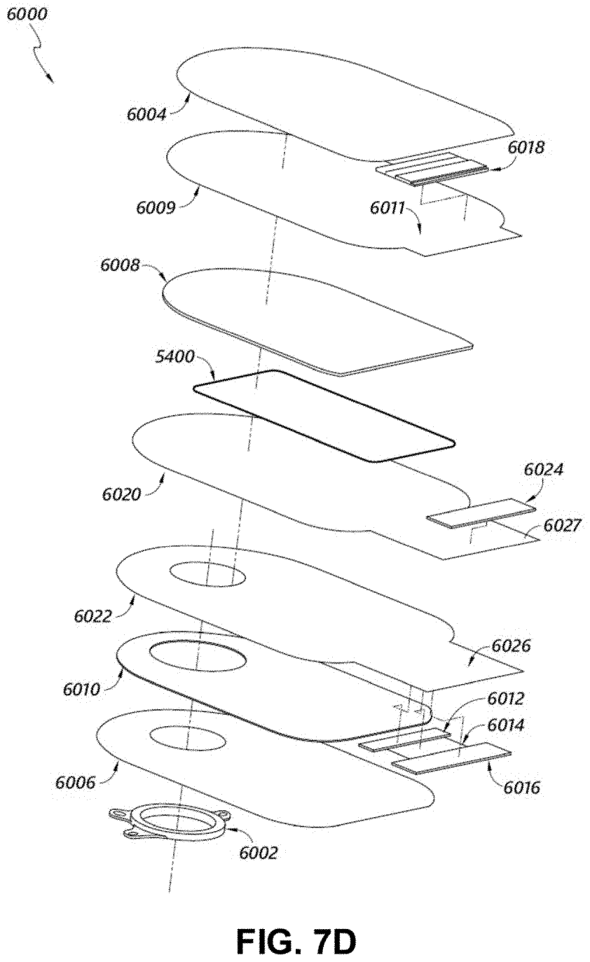

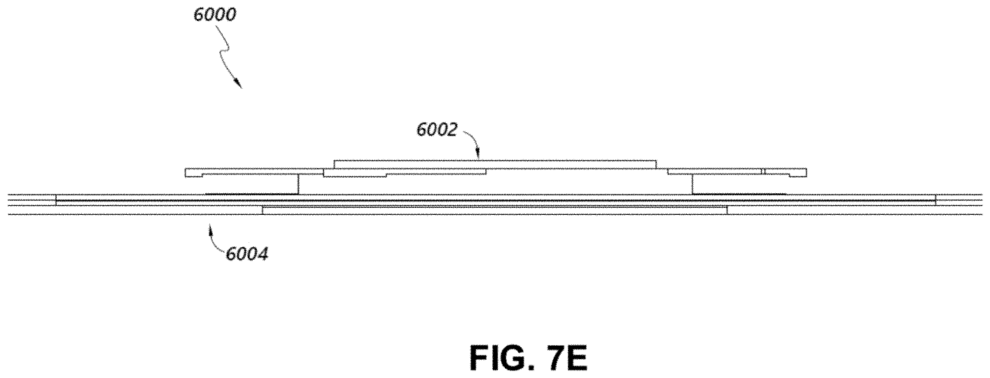

FIGS. 7A-7E illustrate an example ostomy bag 6000 that incorporates a sensor layer 5400 (see FIG. 6), which may be used in conjunction with a monitoring device, such as the monitoring device discussed with reference to FIGS. 1A-4. The bag 6000 can have rounded corners. The bag 6000 can include a plurality of layers. FIG. 7A illustrates an example of the ostomy bag 6000 with internal layers shown in dashed lines and FIG. 7D illustrates in an exploded view the various layers of the bag 6000.

The bag 6000 can include a wafer interface 6002 that can couple with an ostomy wafer, for example, the wafer 5300 at the coupler 5304. The bag 6000 can include a first set of layers, such as a top first layer 6004 and a bottom first layer 6006. The first set of layers can optionally be made of the same material. The bottom first layer 6006 can include an opening to accommodate the stoma. The bottom first layer 6006 can also be coupled to the wafer interface 6002 around the opening on the layer 6006.

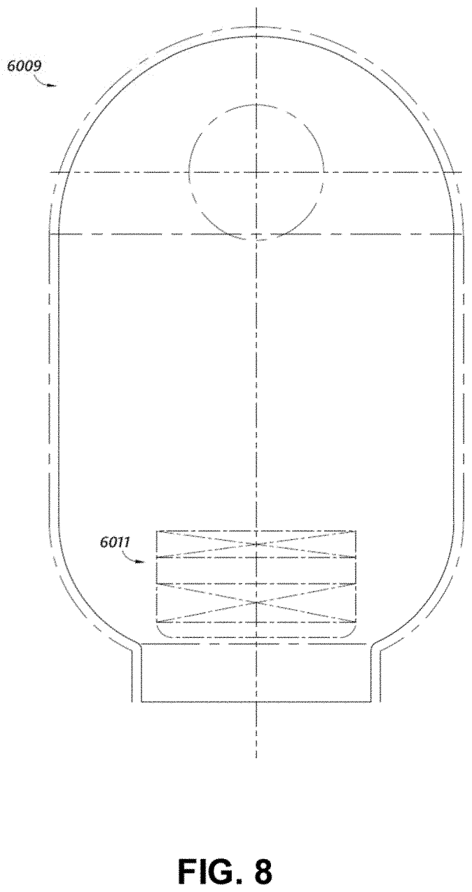

The bag 6000 can include a second set of layers that are sandwiched between the first set of layers. The second set of layers can provide insulation between the sensor layer 5400 and the patient's body or between the sensor layer 5400 and the ambient environment. The second set of layers can optionally be made from the same material. The second set of layers can include a top second layer 6008 and a bottom second layer 6010. A plastic film 6009, such as a polymer film, can be sandwiched between the top first layer 6004 and the top second layer 6008. As shown in FIGS. 7D and 8, the film 6009 can have an area 6011 on its top surface that is configured to receive (for example, via adhesives, welding, or otherwise) a hook or loop portion 6018 (such as a hook portion) of a Velcro connector.

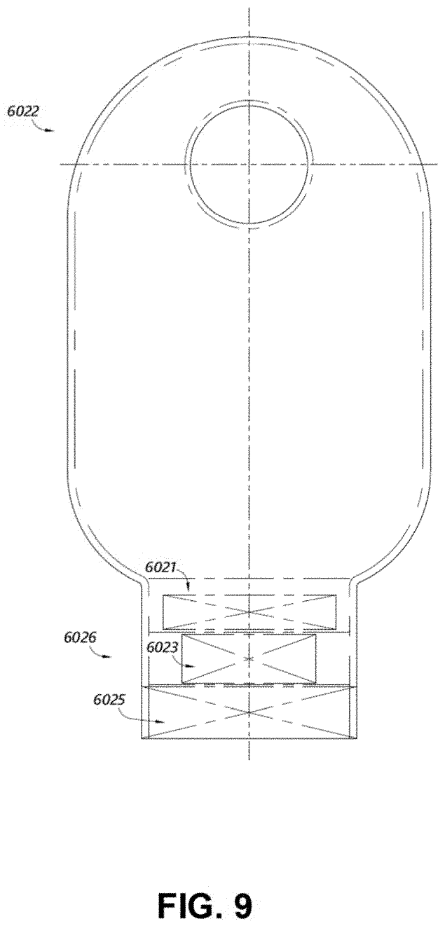

The bag 6000 can include a third set of layers that are sandwiched between the second set of layers. The third set of layers can optionally be made from the same material. The third set of layers can include a top third layer 6020 and a bottom third layer 6022. As shown in FIGS. 7D and 9, the bottom third layer 6022 can have on its bottom surface a first area 6021 configured to be coupled to a loop or hook portion 6012 (such as a loop portion) of the Velcro connector that is complementary to the hook or loop portion 6018, a second area 6023 configured to be coupled to a metallic tape 6014, and a third area 6025 configured to be coupled to a bottom drain tab 6016 at or near a draining location of the bag 6000. The Velcro hook or loop 6012, the metallic tap 6014, and the bottom drain tab 6016 can be adjacent to one another. The first area 6021, second area 6023, and third area 6025 can be located on an extension 6026. The top third layer 6020 can have a corresponding extension 6026.

The sensor layer 5400 can be on top of the top third layer 6020 and between the top second layer 6008 and the top third layer 6020. The sensor layer 5400 can be coupled to the top third layer 6020 via an adhesive or otherwise. The sensor layer 5400 can also optionally be placed between other layers or at other locations of the ostomy bag. The top third layer 6020 can include on its top surface an area 6027 configured to be coupled to a top drain tab 6024. When assembled, as shown in FIGS. 7B-7C, the top drain tab 6024 and the bottom drain tab 6016 are aligned so as to sandwich the top and bottom third layers 6020, 6022 between the two tabs 6024, 6016. When assembled, the hook or loop portion 6018 and the loop or hook portion 6012 can be located on opposite sides of the bag 6000 but adjacent to each other. As a result, when the extensions 6026 of the top and bottom third layers 6020, 6022 are folded onto a remainder of the bag 6000, the two portions 6018, 6012 can mate with each other to secure the extensions 6026 onto the remainder of the bag 6000. Optionally, the Velcro connector can be replaced by any reusable or quick-release connectors, for example, magnets, conductive Velcro connector, buttons, suitable adhesives, or otherwise.

The folding of the extensions 6026 can close or seal the drain opening of the bag 6000. The extensions 6026 can have a length of about 30 mm to about 80 mm, or about 40 mm to about 70 mm, or about 55 mm to about 60 mm. The extensions 6026 can have a width of about 50 mm to about 110 mm, or about 65 mm to about 95 mm, or about 80 mm. When folding the extensions 6026, the top and bottom drain tabs 6024, 6016 can first be folded so that the top and bottom drain tabs 6024, 6016 are on an opposite side of the third set of layers from the metallic tape 6014. The top and bottom drain tabs 6024, 6016 and the metallic tape 6014 are then folded so that the top and bottom drain tabs 6024, 6016 and the metallic tape 6014 are on an opposite side of the third set of layers from the hook or loop portion 6018. To resealably close the drain opening, the hook or loop portion 6018 is folded over to bond with the loop or hook portion 6012.

When the extensions 6026 are folded, a length of the bag 6000 can be about 150 mm to about 250 mm, or about 180 mm to about 220 mm, or about 205 mm. When the extensions 6026 are folded, a width of the bag 6000 can be about 100 mm to about 160 mm, or about 110 mm to about 150 mm, or about 120 mm to about 140 mm, or about 135 mm.

When the extensions 6026 are folded to close the drain opening, the metallic tape 6014 can be in contact (which may be indirectly through the plurality of layers of the bag 6000) with the two capacitive sensors 5404 near the drain opening on the side of the sensor layer 5400 facing away from the patient. When the extensions 6026 are unfolded, the metallic tape 6014 can be out of contact with those two capacitive sensor 5404. The capacitive sensors 5404 can output different signals between when the metallic tape 6014 is contacting the sensors 5404 and when the metallic tape 6014 does not contact the sensors 5404 as the metallic tape 6014 has a different capacitance value than the bag material and/or the fluid inside the bag. A change in the capacitance readings in at least one of the two capacitive sensors 5404 can be indicative of detection of a draining event. Having two capacitive sensors 5404 or two electrodes for detecting whether the metallic tape 6014 is in contact can provide redundancy in case one of the two sensors fail or malfunction.

Optionally, the capacitive sensors 5404 on the top side of the sensor layer 5400 can be replaced by any distance sensors capable of detecting a distance of the metallic tape 6014 from the distance sensor so as to detect whether the draining opening of the bag 6000 has been opened. The distance sensor can also optionally be configured to detect release of gas from the stoma as the bag expands when there is an output of gas. Optionally, the capacitive sensors 5404 on the side of the sensor layer 5400 facing away from the patient can be replaced by a magnetic sensor, which can detect whether the metallic tape 6014 is magnetically attracted to the magnetic sensor (such as AMR (Anisotropic Magneto Resistive) sensor, which can be digital or analog) or detached from the magnetic sensor so as to determine whether a draining event has occurred. The bag sensor layer can include a magnetic field sensor detecting in changes in the magnetic field as the drain opening is open or closed. Optionally, the draining detection sensor can be incorporated into the Velcro connector (for example, conductive hook and loop connector) directly so that whether the hook and loop portions are connected or disconnected can be indicative of whether a draining event has occurred.

The ostomy bag can have at least two or more layers. The number of layers, the arrangement of layers, and the type of materials can be varied. One or more layers of the materials can be coupled together using any suitable coupling and/or bonding mechanisms, such as adhesives, welding, or otherwise.

Another difficulty in accurately detecting electronically the level of the fill of an ostomy bag is the residue problem. When the stoma output is more viscous, such as when the output includes feces or other more solids, the more viscous components can cling to the inner surface(s) of the bag. The solids drying out (“pancaking”) on the inner surface of the bag can result in misleading or false level reading and thus volume calculation. The dried solids can cause opposing inner surfaces of the bag to be stuck, obstructing entry and/or downward movement of the output discharged or infused into the bag. Prolonged exposure of the stoma to the “pancaked” output can also cause infection.

Recalibration of the capacitive sensors to update the baseline values of those sensors can help reduce the influence of the residue problem in the level and volume determination. Alternatively and/or additionally, more capacitive sensors (such as greater than 12 capacitive sensors, for example, from about 36 to about 48 capacitive sensors) can be used on the sensor layer of the ostomy bag to alleviate the effect of residue problem on the level readings. More capacitive sensors and/or increased capacitive sensor density can provide greater resolution in the sensor reading, which can help detect a residue or “pancaked” output as the residue can have a more random shape than the content of the output that has fallen to the bottom of the bag. Accordingly, more capacitive sensors and/or increased capacitive sensor density can improve the accuracy in predicting the volume of the output. In some configurations, the sensor layer including more than 12 capacitive sensors may also include fewer than 64 temperature sensors (such as about 20 temperature sensors).

Alternatively and/or additionally, the inner surface of the ostomy bag layer can be coated with a material, which can reduce the friction coefficient of the inner surface of the ostomy bag and guide the stoma output toward the bottom of the bag. For example, the material can be hydrophilic or hydrophobic. Coating of the material can be achieved through a variety of ways, such as spraying, dipping, or otherwise. The coating can be effective throughout a life cycle of the bag and can be more convenient than having to wash the inner surface of the bag with lubricating materials each time after the bag is drained. The coating can also be more convenient than applying an adhesive layer of hydrophilic lubricating material to the inner surface of the bag, wherein the hydrophilic layer requires substantial moisture to become hydrated and lubricious, so the beneficial effects of reducing the residue problem the may not be realized unless the output discharged into the bag is sufficiently liquid to activate the hydrophilic coating material.

The coating of the material may be biocompatible, and may also be non-biocompatible. The biocompatible coating may be a coating that is inert to biological material, has minimal toxic or injurious effect on biological systems, or is approved for use in biomedical applications. For example, the coating may be a type of silicone oil that is graded for medical applications. The non-biocompatible material may be any other type of coating. For example, the non-biocompatible coating may be a fluorinated silicone oil or flourosilicone oil.

The coating of the material may also be used in other medical applications and devices. For example, the coating may be used to coat the inner surface of any medical bag, medical bottle (for example, a bottle containing a viscous medication), medical container, the surface of a catheter, injection needles, surgical tools, or other medical devices in which a lower friction coefficient and/or where lubrication would be desirable.

Terminology

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a hardware processor comprising digital logic circuitry, a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. A computing environment can include any type of computer system, including, but not limited to, a computer system based on a microprocessor, a mainframe computer, a digital signal processor, a portable computing device, a device controller, or a computational engine within an appliance, to name a few.

The steps of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC.

Conditional language used herein, such as, among others, “can,” “might,” “may,” “for example,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others.

1. A self-contained analysis device configured to be placed within an effluent container, the device comprising:
a power source;
one or more hardware processors;
one or more sensors configured to detect parameters associated with a fluid of a patient;
a wireless transmitter; and
a housing configured to enclose the power source, the one or more hardware processors, and the wireless transmitter, and to position the one or more sensors to be in contact with the fluid, the housing configured to allow the system to be removably placed within the effluent container,
wherein the one or more hardware processors are configured to output a signal based on sensor data from the one or more sensors, and
wherein the wireless transmitter is configured to transmit the signal to an external device.

2. (canceled)

3. The device of claim 1, wherein the effluent container comprises an ostomy bag, urinary catheter, specimen container, or toilet bowl, or wherein the fluid of a patient comprises at least one of urine or fecal waste.

4. (canceled)

5. The device of claim 1, wherein the housing comprises a waterproof casing, wherein the one or more sensors are at least partially enclosed within the waterproof casing.

6. (canceled)

7. (canceled)

8. The device of claim 1, comprising a filter configured to filter solids from the fluid of the patient.

9. The device of claim 1, comprising one or more microfluidic channels configured to transmit the fluid to the one or more sensors.

10. The device of claim 1, comprising an agitator configured to agitate the fluid, wherein the agitator is configured to cause the fluid to pass through the one or more sensors.

11. (canceled)

12. The device of claim 1, wherein the one or more sensors comprise an electrochemical sensor configured to measure at least one of sodium, glucose, or potassium level in the fluid.

13. (canceled)

14. (canceled)

15. (canceled)

16. (canceled)

17. (canceled)

18. (canceled)

19. A self-contained system for analyzing the contents of an ostomy bag, the system comprising:
an ostomy bag;
a monitoring device comprising:
one or more sensors configured to detect parameters associated with effluent contained in the ostomy bag; and
a housing configured to enclose a wireless transmitter and the one or more sensors, the housing configured to place the one or more sensors in contact with the effluent, the housing configured to allow the system to be placed within the ostomy bag,
wherein the wireless transmitter is configured to transmit sensor data to an external device; and
at least one hardware processor in communication with the at least one or more sensors configured to:
receive sensor data from the one or more sensors;
determine at least one effluent parameter based on the sensor data;
analyze the at least one effluent parameter to determine a parameter characteristic; and
transmit an alert associated with the sensor data to a clinician device based on the parameter characteristic.

20. The system of claim 19, wherein the housing comprises a waterproof casing and the one or more sensors are at least partially enclosed in the waterproof casing.

21. (canceled)

22. (canceled)

23. The system of claim 19, wherein the monitoring device comprises a filter configured to filter solids from the effluent.

24. The system of claim 19, wherein the monitoring device comprises one or more microfluidic channels configured to transmit effluent to the one or more sensors.

25. (canceled)

26. (canceled)

27. The system of claim 19, wherein the one or more sensors comprise an electrochemical sensor configured to measure at least one of sodium, glucose, or potassium level.

28. (canceled)

29. (canceled)

30. The system of claim 19, wherein the at least one hardware processor is configured to transmit the alert in response to determining that the parameter characteristic has exceeded a threshold criteria.

31. The system of claim 29, wherein the threshold criteria comprises a rate of change of the parameter characteristic over a period of time.

32. (canceled)

33. (canceled)

34. (canceled)

35. (canceled)

36. (canceled)

37. The system of claim 19, wherein the housing is configured to enclose a power source.

38. (canceled)

39. A self-contained monitoring system which analyzes the contents of an ostomy bag, the system comprising:
one or more sensors configured to detect parameters associated with effluent contained in the ostomy bag; and
a housing configured to enclose a wireless transmitter and the one or more sensors, the housing configured to position the one or more sensors to be in contact with the effluent, the housing configured to allow the system to be placed within the ostomy bag,
wherein the wireless transmitter is configured to transmit sensor data to an external device.

40. The system of claim 39, wherein the housing comprises a waterproof casing and the one or more sensors are at least partially enclosed in the waterproof casing.

41. (canceled)

42. The system of claim 39, comprising a filter configured to filter solids from the effluent.

43. The system of claim 39, comprising one or more microfluidic channels configured to transmit effluent to the one or more sensors.

44. (canceled)

45. (canceled)

46. The system of claim 39, wherein the one or more sensors comprise one or more electrochemical sensors.

47. (canceled)

48. The system of claim 39, comprising one or more hardware processors, wherein the one or more hardware processors is configured to analyze the sensor data to determine if the sensor data passes a threshold criteria.

49. The system of claim 48, wherein the one or more hardware processors are configured to output an alert in response to the sensor data passing the threshold criteria.

50. The system of claim 48, wherein the threshold criteria comprises a rate of change of a parameter associated with the sensor data over a period of time.

51. (canceled)

52. (canceled)

53. (canceled)

54. (canceled)

55. (canceled)

56. The system of claim 39, wherein the housing is configured to enclose a power source.

57. (canceled)
